(19) United States
(12) Patent Application Publication
(10) Pub. No.: US 2023/0002837 A1
(43) Pub. Date: Jan. 5, 2023

(54) METHODS AND COMPOSITIONS FOR PROVIDING IDENTIFICATION AND/OR TRACEABILITY OF BIOLOGICAL MATERIAL

(71) Applicant: INDEX BIOSYSTEMS INC., Toronto (CA)

(72) Inventors: Michael BORG, St. Catharines (CA); Jeremy N. FRIEDBERG, Guelph (CA)

(21) Appl. No.: 17/780,030

(22) PCT Filed: Nov. 26, 2020
(86) PCT No.: PCT/CA2020/051622

§ 371 Date: May 26, 2022

Related U.S. Application Data
(60) Provisional application No. 62/940,587, filed on Nov. 26, 2019.

(51) Int. Cl. C12Q 1/6888; C12Q 1/70; G16B 50/30; C12Q 1/6806

(52) U.S. Cl. CPC C12Q 1/6888; C12Q 1/701; G16B 50/30; C12Q 1/6806; C12Q 2600/158

(57) ABSTRACT

Provided herein are methods and compositions for providing identification and/or traceability of biological materials. In certain embodiments, methods are provided including steps of: determining a sequence of at least one unique identifier sequence in the genomic DNA of a biological entity; validating identification of the biological entity by verifying presence of the unique identifier sequence in the genomic DNA and comparing the sequence of the unique identifier sequence with a database to confirm uniqueness; providing an indication of acceptability to produce a biological material from the biological entity; and inputting the unique identifier sequence into a database entry of the database and associating the unique identifier sequence with identification and/or tracking information; thereby providing traceability by reading the unique identifier sequence and retrieving the corresponding database entry to obtain the identification and/or tracking information. Oligonucleotides, cassettes, and compositions for providing identification and/or traceability of biological materials are also provided.

### FIELD OF INVENTION

The present invention relates generally to the identification and/or tracking of biological materials. More specifically, the present invention relates to methods and agents for the identification and/or tracking of biological materials using nucleic acid.

### BACKGROUND

The food system has reached unprecedented levels of distribution efficiency and production output. This evolution has afforded great benefits to the public in the form of cost-reduction and variety; however, serious deficiencies remain which expose risk to public health, industry and innovation. Traceability is one of the primary techniques for the effective governance and management of these challenges.

The limitations of current food and beverage traceability systems are primarily exposed through contamination events. When these events occur, it may take months to trace the effected products to their source-of-origin. Clonally propagated products may add additional challenges to source-of-origin identification because they lack genetic variation. Transformed and mixed-item products may also be problematic for source-of-origin identification because they require that existing traceability best practices are followed throughout the supply chain. Shortcomings in the ability to promptly and affordably trace these products poses significant risk to consumer safety; have caused material financial losses for stakeholders; and resulted in profound damage to the reputation of effected industries.

In 2015, the World Health Organization (WHO) completed a 10 year-long initiative estimating the global burden of foodborne disease. This initiative found the “ . . . global burden of foodborne disease . . . was 33 (95% UI 25-46) million DALYs in 2010; 40% of the foodborne disease burden was among children under 5 years of age.” (p. 11). DALY stands for disability adjusted life years. It can be thought of as one lost year of healthy life. The estimations made by this study were limited by data gaps. Improved surveillance and laboratory capacity were noted as required for more accurate estimation. Surveillance needs were further identified by the source attribution task force (SATF).

The SATF was among numerous task forces commissioned for this initiative. Their mandate was to estimate the effects of particular attribution points on disease transmission. FIG. 1 (adapted from WHO, 2015, p.101) illustrates major points of attribution. FERG, the reference group for this study, determined that for the purposes of the study, the most simple point-of-attribution is at the end of the transmission chain—i.e. human contact. This simplicity is a property of the limitations of existing traceability practices. FERG also notes (p. 100) that for risk management, other points of attribution may be more appropriate—e.g. primary production. FERG identifies surveillance for reservoir level attribution as desirable.

Modern techniques for food traceability in the food and beverage supply-chain typically begin with a grower's harvest or within a production facility. Products are often tracked at the case level—a case contains many items. Occasionally a physical barcode is applied to each item. A Global Trade Item Number (GTIN) and Global Location Number (GLN) is ideally associated with a case. A Serial Shipping Container Code (SSCC) may be created for a pallet—a collection of cases. These traceability techniques are typically prescribed by a standard, and for fresh food, that is often the GS1 Standard. As pallets make their way through the supply chain, the aforementioned identifiers found on barcodes are used in conjunction with key data elements (KDEs) recorded for critical tracking events (CTEs). A CTE might describe product disposition from a grower to a packer/shipper. There is a commonly used aphorism that suggests each supply-chain stakeholder should be able to trace a product “one-step forward and one-step back”. Unfortunately, that requirement has proven to be inadequate in many ways.

Once a food item reaches the point-of-sale, it may have been transformed or comingled with other items from disparate producers—e.g. fruit salad. Often, as soon as an item separates from its original case, or item-level identifier, it is often impossible to trace the item back to the producer. As seen with the recent romaine outbreak, it took investigators over a month to pinpoint the source of the contamination, because they did not have source-of-origin information (FDA, 2019, p. 1), even though the vast majority of production occurs in the U.S. southwest. As a result, the FDA has urged “ . . . the entire leafy greens supply chain to adopt traceability best practices and state-of-the-art technology to assure quick, accurate and easy access to key data elements from farm to fork when leafy greens are involved in a potential recall or outbreak.” (FDA, 2019, p. 8). The costs associated with this outbreak are still being uncovered. However, other contamination events are well known.

The spinach recall from 2006 was linked to five deaths and approximately 200 life-threatening illnesses in 26 states. It caused approximately $500 million in financial damage (GS1, 2013, p. 3). More generally, “ . . . government agencies have also expressed concern over the health and financial impact of recent food recalls, as foodborne illnesses impact 48 million people a year and cost the United States $152 billion in healthcare costs every year.” (GS1, 2013, p. 2). Whole-chain traceability, which can be understood as seed-to-sale tracking, was found to reduce the total amount of product recalled to 12% of cases for Frontera Produce's cilantro recall. McKinsey found that a 25% improvement in recall precision could save the fresh foods industry $250-$275 million each year (GS1, 2013, p. 10).

Whole-chain traceability has lacked an effective form of item-level identification, and has lacked guarantees about source-of-origin. Existing methods for item-level identification typically rely on physical branding (lasers), Radio Frequency identifier (RFID), and barcoding—i.e. external physical identifiers. There are scaling challenges associated with these techniques as well. Each item requires a physical identifier and has cost associated with its production. Additionally, there is risk of erroneous reads and/or malicious tampering risks inherent in their use—e.g. stickers fall off or are removed.

Food contamination, such as E. coli and/or salmonella contaminations affecting the food supply, are a threat to public health and rapid action to identify and stem source(s) of contamination is highly desirable. There is a long-felt unmet need in the field for reliable, cost-effective, and/or rapid strategies for enhancing the traceability of products in the food supply. Traceability of biological entities and/or biological materials is desirable not only in the agriculture and food industries, but is also sought-after in a wide variety of industries and fields dealing with biological entities and/or biological materials containing or derived therefrom. Alternative, additional, and/or improved methods and/or compositions for providing identification and/or traceability of biological entities and/or biological materials is desirable.

### SUMMARY OF INVENTION

Provided herein are methods and compositions for providing identification and/or traceability of biological materials. In certain embodiments, methods as described herein may make use of a unique identifier sequence (also referred to herein as a DNA unique identifier sequence), which is exogenously introduced into the genome of a biological entity, in order to provide for identification and/or traceability of the biological entity and/or biological materials comprising the biological entity and/or biological materials produced from the biological entity and containing genomic DNA therefrom. In certain embodiments, the unique identifier sequence may be from a randomized pool of sequences. In certain embodiments, a database may be maintained linking unique identifier sequences with corresponding identification and/or tracking information. Also provided herein are oligonucleotide constructs and cassettes comprising one or more unique identifier sequences for use in providing identification and/or traceability of biological materials. In certain embodiments, oligonucleotide constructs and/or cassettes may comprise particular arrangements of primer annealing sequence(s), which may be for amplification of the unique identifier sequence(s), sequencing of the unique identifier sequence(s), or both. In certain embodiments, methods and compositions as described herein may be used for providing food traceability, and may allow for quick response and/or food recall in the event of a contamination, for example.

In an embodiment, there is provided herein a method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the biological material.

In another embodiment of the above method, the biological material may comprise a plant-based material, a fungus-based material, an animal-based material, a virus-based material, or a bacterial-based material.

In certain embodiments, the biological material may comprise a fungus-based material. In certain embodiments the biological material may comprise a yeast. In certain embodiments, the yeast may, optionally, be sporulated (i.e. the biological material may comprise a yeast spore). In certain embodiments, the yeast may be added to, mixed, or otherwise associated with a product for which identification and/or tracking is desired, such as a food ingredient or a food product.

In another embodiment, there is provided herein a method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and comparing the sequence of the DNA unique identifier sequence with a database to confirm that the DNA unique identifier sequence is not already used in the database;
providing an indication of acceptability to produce a biological material from the biological entity, the biological material comprising genomic DNA from the biological entity; and
inputting the sequence of the at least one DNA unique identifier sequence into a database entry of the database, and associating the DNA unique identifier sequence with identification and/or tracking information for the biological material;

thereby providing traceability of the biological material by reading the DNA unique identifier sequence in the biological material and retrieving the corresponding database entry providing the identification and/or tracking information for the biological material.

In another embodiment of the above method, the method may further comprise inserting at least one DNA unique identifier sequence within the genomic DNA of a biological entity, or modifying a pre-existing identifier sequence within the genomic DNA of a biological entity by gene editing to create a DNA unique identifier sequence within the genomic DNA of the biological entity, thereby providing identification thereof.

In yet another embodiment of any of the above method or methods, the method may further comprise providing the at least one DNA unique identifier sequence for the insertion within the genomic DNA of the biological entity.

In still another embodiment of any of the above method or methods, the biological material may comprise a plant-based material, a fungus-based material, an animal-based material, a virus-based material, or a bacterial-based material.

In yet another embodiment of any of the above method or methods, the biological entity may comprise a plant cell, a fungal cell, an animal cell, a virus, or a bacterial cell.

In another embodiment of any of the above method or methods, the biological material, the biological entity, or both, may comprise a fungal-based material or a fungal cell. In certain embodiments, the biological material, the biological entity, or both, may comprise a yeast. In certain embodiments, the yeast may, optionally, be sporulated (i.e. may comprise a yeast spore).

In still another embodiment of any of the above method or methods, producing a biological material from the biological entity may comprise propagating the biological entity.

In another embodiment of any of the above method or methods, the DNA unique identifier sequence may be from a randomized pool of DNA unique identifier sequences.

In yet another embodiment of any of the above method or methods, reading the DNA unique identifier sequence in the biological material and retrieving the corresponding database entry may comprise:
receiving or providing a sample comprising genomic DNA from the biological material;
amplifying the at least one DNA unique identifier sequence within the genomic DNA from the biological material and sequencing the DNA unique identifier sequence; and
comparing the DNA unique identifier sequence to the database and retrieving the database entry corresponding with the DNA unique identifier sequence, the database entry providing identification and/or tracking information for the biological material.

In still another embodiment of any of the above method or methods, the DNA unique identifier sequence may comprise a unique nucleotide sequence inserted into an intergenic region of the genomic DNA.

In yet another embodiment of any of the above method or methods, the DNA unique identifier sequence may comprise a sequence of up to about 1500 nt in length; up to about 1000 nt in length; about 200 nt to about 600 nt in length; about 200 nt to about 400 nt in length; or about 400 nt to about 600 nt in length.

In another embodiment of any of the above method or methods, the DNA unique identifier sequence may be flanked by one or more primer annealing sequences for PCR amplification of the DNA unique identifier sequence, sequencing of the DNA unique identifier sequence, or both.

In yet another embodiment of any of the above method or methods, the biological material may comprise a food.

In still another embodiment of any of the above method or methods, the identification and/or tracking information of the database entry may comprise supply chain information for the biological material. In certain embodiments, the supply chain information may comprise supply chain information for a food, agricultural, pharmaceutical, retail, textile, commodity, chemical, or other supply chain item with which the biological material may be associated.

In another embodiment of any of the above method or methods, the identification and/or tracking information of the database entry may comprise source-of-origin information for the biological material.

In yet another embodiment of any of the above method or methods, the identification and/or tracking information of the database entry may comprise grower, region, batch, lot, date, or other relevant supply chain information, or any combinations thereof.

In still another embodiment of any of the above method or methods, a cassette may be incorporated into the genomic DNA, wherein the cassette may comprise the DNA unique identifier sequence flanked by one or more primer annealing sequences for PCR amplification of the DNA unique identifier sequence, sequencing of the DNA unique identifier sequence, or both.

In another embodiment of any of the above method or methods, the DNA unique identifier sequence may be a random sequence derived from a randomized pool of nucleic acid sequences of up to about 1500 nt in length; up to about 1000 nt in length; about 200 nt to about 600 nt in length; about 200 nt to about 400 nt in length; or about 400 nt to about 600 nt in length.

In another embodiment, there is provided herein an oligonucleotide comprising a DNA unique identifier sequence flanked by one or more primer annealing sequences for PCR amplification of the DNA unique identifier sequence, sequencing of the DNA unique identifier sequence, or both.

In another embodiment of the above oligonucleotide, the DNA unique identifier sequence may comprise a random sequence of up to about 1500 nt in length; up to about 1000 nt in length; about 200 nt to about 600 nt in length; about 200 nt to about 400 nt in length; or about 400 nt to about 600 nt in length.

In another embodiment, there is provided herein a cassette comprising any of the oligonucleotide or oligonucleotides as described herein.

In still another embodiment, there is provided herein a cell or virus comprising any of the oligonucleotide or oligonucleotides as described herein, or any of the cassette or cassettes as described herein, incorporated into the genome of the cell or virus.

In another embodiment, there is provided herein a cell or virus comprising a DNA unique identifier sequence incorporated into the genome of the cell or virus.

In another embodiment of any of the above cells or viruses, the DNA unique identifier sequence may be incorporated into an intergenic region of the genomic DNA of the cell or virus.

In still another embodiment of any of the above cells or viruses, the cell may be a plant cell, a fungal cell, an animal cell, or a bacterial cell.

In another embodiment, the cell may be a fungal cell, such as a yeast cell.

In another embodiment, there is provided herein a kit comprising any one or more of:
a DNA unique identifier sequence;
a randomized pool of DNA unique identifier sequences;
any of the oligonucleotide or oligonucleotides as described herein;
any of the cassette or cassettes as described herein;
one or more primer pairs for amplifying and/or sequencing a DNA unique identifier sequence;
a buffer;
a polymerase; or
instructions for performing any of the method or methods as described herein.

In another embodiment, there is provided herein a method of identifying a biological material, the method comprising:
receiving at a computing device a DNA-unique identifier sequence (DUID) extracted from a known biological material;
searching at the computing device a DUID database storing a plurality of DUIDs in association with respective biological material information for a match to the received DUID;
if the search of the DUID database fails to provide a match to the received DUID, storing in the DUID database the received DUID in association with biological material information associated with the known biological material;
subsequent to storing the received DUID and with information associated with the known biological material in the DUID database, receiving at the computing device a query DUID extracted from an unknown biological material;
searching at the computing device the DUID database for a match to the received query DUID; and
if the search of the DUID provides a match to the received query DUID, returning in response to the received query DUID the biological information stored in association with the DUID matching the query DUID.

In another embodiment of the above method, searching the DUID database for a match to the received DUID may comprise:
searching the DUID database for an exact match to the received DUID; and
if an exact match is not found, performing an alignment/identity search for DUIDs stored in the DUID database that are a close match to the received DUID.

In still another embodiment of any of the above method or methods, searching the DUID database for a match to the query DUID may comprise:
searching the DUID database for an exact match to the query DUID; and
if an exact match is not found, performing an alignment/identity search for DUIDs stored in the DUID database that are a close match to the query DUID.

In yet another embodiment of any of the above method or methods, the method may further comprise:
if the search provides a close match to the query DUID, storing the query DUID in association with the DUID that is a close match to the query DUID.

In another embodiment, there is provided herein a computing system for identifying a biological material, the system comprising:
a processing unit capable of executing instructions; and
a memory unit storing instructions, which when executed by the processing unit configure the computing system to perform any of the method or methods as described herein.

In another embodiment, there is provided herein a computer readable memory, having instructions stored thereon, which when executed by a processing unit of a computing system configure the system to perform any of the method or methods described herein.

In another embodiment, there is provided herein a method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
decoding or decrypting identification and/or tracking information for the biological material stored in the DNA unique identifier sequence.

In another embodiment, there is provided herein a method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and decoding or decrypting identification and/or tracking information stored in the DNA unique identifier sequence to verify the DNA unique identifier sequence; and
providing an indication of acceptability to produce a biological material from the biological entity, the biological material comprising genomic DNA from the biological entity;

thereby providing traceability of the biological material by reading the DNA unique identifier sequence in the biological material and decoding or decrypting information stored in the DNA unique identifier sequence, providing identification and/or tracking information for the biological material.

In still another embodiment, there is provided herein a method of identifying a biological material, the method comprising:
receiving at a computing device a DNA-unique identifier sequence (DUID) extracted from an unknown biological material; and
decoding or decrypting identification and/or tracking information for the unknown biological material stored in the DNA unique identifier sequence.

In another embodiment, there is provided herein a cassette comprising a DNA unique identifier sequence, the DNA unique identifier sequence flanked by at least one 5′ primer annealing sequence and at least one 3′ primer annealing sequence for amplification of the DNA unique identifier sequence, sequencing of the DNA unique identifier sequence, or both.

In another embodiment of the above cassette, the DNA unique identifier sequence may be flanked by two 5′ primer annealing sequences and two 3′ primer annealing sequences to allow for amplification of the DNA unique identifier sequence by nested PCR.

In still another embodiment of any of the above cassette or cassettes, the two 5′ primer annealing sequences may be partially overlapping; the two 3′ primer annealing sequences may be partially overlapping; or both.

In still another embodiment of any of the above cassette or cassettes, the cassette may further comprise a sequencing primer annealing sequence located 5′ to the DNA unique identifier sequence for sequencing of the DNA unique identifier sequence.

In yet another embodiment of any of the above cassette or cassettes, the sequencing primer annealing sequence may be positioned between two 5′ primer annealing sequences.

In another embodiment of any of the above cassette or cassettes, the sequencing primer annealing sequence may at least partially overlap with one or both of the two 5′ primer annealing sequences.

In yet another embodiment of any of the above cassette or cassettes, the two 5′ primer annealing sequences may be partially overlapping, and at least a portion of the sequencing primer annealing sequence may be positioned at the overlap.

In another embodiment of any of the above cassette or cassettes, the cassette sequence may be up to about 1500 nt in length; up to about 1000 nt in length; about 200 nt to about 600 nt in length; about 200 nt to about 400 nt in length; or about 400 nt to about 600 nt in length.

In still another embodiment of any of the above cassette or cassettes, the primer annealing sequences may not be naturally occurring in the genome of a target biological entity.

In another embodiment, there is provided herein a composition comprising a plurality of any of the cassette or cassettes as described herein, each cassette comprising the same primer annealing sequences, and each cassette comprising a randomized DNA unique identifier sequence.

In still another embodiment, there is provided herein a composition comprising a plurality of any of the cassette or cassettes as described herein, each cassette comprising the same primer annealing sequences and the same sequencing primer annealing sequence, and each cassette comprising a randomized DNA unique identifier sequence.

In yet another embodiment, there is provided herein a method for providing traceability of biological material, said method comprising:
inserting at least one DNA unique identifier sequence within the genomic DNA of a biological entity for use in preparing the biological material.

In another embodiment of the above method, the DNA unique identifier sequence may be inserted as any of the cassette or cassettes as described herein.

In another embodiment of any of the above method or methods, the method may further comprise a step of determining the sequence of the least one DNA unique identifier sequence within the genomic DNA of the biological entity.

In another embodiment of any of the above method or methods, the method may further comprise a step of validating identification of the biological entity by: verifying presence of the DNA unique identifier sequence in the genomic DNA; and comparing the sequence of the DNA unique identifier sequence with a database to confirm that the DNA unique identifier sequence is not already used in the database.

In still another embodiment of any of the above method or methods, the method may further comprise a step of:
producing the biological material from the biological entity, the biological material comprising genomic DNA from the biological entity; and/or
providing an indication of acceptability to produce the biological material from the biological entity, the biological material comprising genomic DNA from the biological entity.

In still another embodiment of any of the above method or methods, the method may further comprise a step of inputting the sequence of the at least one DNA unique identifier sequence into a database entry, and associating the DNA unique identifier sequence with identification and/or tracking information for the biological entity and/or biological material.

In yet another embodiment of any of the above method or methods, the method may further comprise a step of:
providing traceability of the biological entity and/or biological material by reading the DNA unique identifier sequence in the biological entity and/or biological material and retrieving the corresponding database entry providing the identification and/or tracking information for the biological entity and/or biological material.

In another embodiment, there is provided herein a plasmid or expression vector comprising any of the oligonucleotide or oligonucleotides or cassette or cassettes as described herein.

In yet another embodiment, there is provided herein a method for providing traceability of a product of interest, said method comprising:
receiving or providing a sample from the product of interest, the sample comprising genomic DNA from a biological material part of, mixed with, or otherwise associated with the product of interest;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the product of interest.

In another embodiment of the above method, the method may comprise introducing or adding any of the biological material or biological materials or biological entity or biological entities as described herein to the product of interest, the biological material or entity comprising at least one DNA unique identifier sequence as described herein as part of its genomic material.

In yet another embodiment of any of the above method or methods, the identification and/or tracking information of the database entry may comprise supply chain information for the product of interest.

In still another embodiment of any of the above method or methods, the product of interest may comprise food, an agricultural product, a pharmaceutical drug, a retail product, textiles, commodities, chemicals, or another supply chain item.

### BRIEF DESCRIPTION OF DRAWINGS

These and other features will become further understood with regard to the following description and accompanying drawings, wherein:

FIG. 1 shows transmission routes identified by the World Health Organization (WHO) in their 2015 report (adapted from WHO, 2015, p.101);

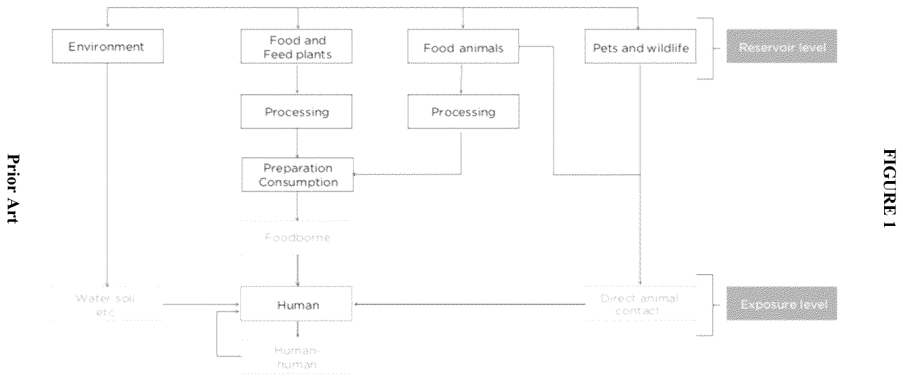

FIG. 2 shows an example of a cassette as described herein including a DUID sequence, and creation thereof as described in Example 1. The depicted sequence is SEQ ID NO: 1;

FIG. 3 shows a global view of the exemplary process for the DUID system described in Example 1;

FIG. 4 shows an example of an identification stage of a DUID system process as described in Example 1;

FIG. 5 shows an example of a validation stage of a DUID system process as described in Example 1;

FIG. 6 shows an example of a read stage of a DUID system process as described in Example 1;

FIG. 7 shows another example of a DUID system and process as described herein;

FIG. 8 shows another example of a DUID system and process as described herein, in which traceability of a biological entity is provided using a DUID and a database/registry;

FIG. 9 shows still another example of a DUID system and process as described herein, in which identification and/or tracking information for a biological material is obtained from a database using a DUID sequence and a database/registry;

FIG. 10 shows another example of a DUID system and process as described herein, in which traceability of a biological entity is provided using a DUID storing tracking and/or identification information;

FIG. 11 shows another example of a DUID system and process as described herein, in which identification and/or tracking information for a biological material is obtained using a DUID sequence storing tracking and/or identification information;

FIG. 12 shows another example of a DUID system and process as described herein, in which identification and/or tracking information for a biological material is obtained using a DUID sequence storing tracking and/or identification information;

FIG. 13 shows additional examples of cassette designs as described herein including a UID (unique identifier) sequence. FIGS. 13(a) shows a dual primer design, 13(b) shows a single primer design, and 13(c) shows a standalone design;

FIG. 14 shows maps of two 370 pb DUID constructs as described in Example 2. A) DUID construct design for PCR and qPCR amplification. Construct is 370 pb. This DUID construct contains 2 forward primers and two reverse primers. There are two identifiers (ID1 and ID2). ID1 is ideal for PCR amplification. ID2 is ideal for qPCR amplification. B) A DUID construct design for loop-mediated isothermal amplification (LAMP) and PCR. This map includes primers for both PCR and LAMP;

FIG. 15 shows detection of YCp-DUID in yeast genomic DNA by end-point PCR as described in Example 2. PCR amplification was performed using (A) YCp-DUID vector and (B) gDNA extracted from BY4743 and (C) yeast strain BY4743 transformed with YCp-DUID vector as templates with DUID recall primers. Reactions were performed using serially diluted DNA template with input quantities of (1) 100 ng, (2) 10 ng, (3) 1 ng, (4) 100 pg, (5) 10 pg, (6) 1 pg, (7) 100 fg and (8) 10 fg and resolved on an 1% agarose gel with GeneRuler™ 100 bp Plus Ready-to-use Ladder as standard;

FIG. 16 shows detection of DUID within yeast total DNA extracts as described in Example 2. Quantitative real-time PCR was performed on serial 10-fold dilutions of YCp vector, ranging from 50 ng-500 ag and used to generate a standard curve (blue line) using MS Excel. Results of a similar qPCR experiment using DNA derived from BY4743 transformed with YCp-DUID vector were plotted (orange bar) and compared with standard curve values to quantify detection of DUID within yeast biomass; and

FIG. 17 shows an example of homology across identifier sequences, which function as a means to identify the version of the DUID, its origin, and subsequence protocols for interacting with the DUID, as further described in Example 2.

### DETAILED DESCRIPTION

Described herein are methods and compositions for providing identification and/or traceability of biological material. It will be appreciated that embodiments and examples are provided for illustrative purposes intended for those skilled in the art, and are not meant to be limiting in any way.

Provided herein are methods and compositions for providing identification and/or traceability of biological materials. In certain embodiments, methods as described herein may make use of a unique identifier sequence (also referred to herein as a DNA unique identifier sequence), which may be exogenously introduced (i.e. inserted/integrated) into the genome of a biological entity, in order to provide for identification and/or traceability of the biological entity and/or biological materials comprising the biological entity and/or biological materials produced from the biological entity and containing genomic DNA therefrom. In certain embodiments, strategies as described herein may benefit from the durability and replicative capacity of nucleic acid such as DNA to provide identification and/or traceability. In certain embodiments, the unique identifier sequence may be from a randomized pool of sequences. In certain embodiments, a database may be maintained linking unique identifier sequences with corresponding identification and/or tracking information.

Also provided herein are oligonucleotide constructs and cassettes comprising one or more unique identifier sequences for use in providing identification and/or traceability of biological materials. In certain embodiments, oligonucleotide constructs and/or cassettes may comprise particular arrangements of primer annealing sequence(s), which may be for amplification of the unique identifier sequence(s), sequencing of the unique identifier sequence(s), or both. In certain embodiments, arrangements of primer annealing sequence(s) may be designed as described herein so as to reduce unintended and/or off-target amplification and/or sequencing events, which may provide for enhanced fidelity and/or reduced errors in identification events, for example.

In certain embodiments, methods and compositions as described herein may be used for providing food traceability, and may allow for quick response and/or food recall in the event of a contamination, for example. Food contamination, such as E. coli and/or salmonella contaminations affecting the food supply, are a threat to public health and rapid action to identify and stem source(s) of contamination is highly desirable. There is a long-felt unmet need in the field for reliable, cost-effective, and/or rapid strategies for enhancing the traceability of products in the food supply. Strategies as described herein may provide for traceability in the food system from source-of-origin to digestion and beyond. Traceability of biological entities and/or biological materials is desirable not only in the agriculture and food industries, but is also sought-after in a wide variety of industries and fields dealing with biological entities and/or biological materials containing or derived therefrom. Accordingly, in addition to food safety, applications in food/seed security, IP tracking, certification (e.g. seed association, Kosher, Halal, etc. . . . ), GMO identification and/or characterization, and/or risk reduction for trade financing are also contemplated herein.

In certain embodiments, food products or ingredients (such as, for example, fruits and vegetables, or other such foodstuffs containing cells) may comprise unique identifier sequence(s) as described herein as part of the genome in at least some cells thereof to provide for identification and/or traceability. In other embodiments, unique identifier sequence(s) as described herein may be part of the genome of one or more biological entities or biological materials comprising cells, and the biological entities or biological materials may be added to, mixed with, or otherwise associated with one or more products for which identification and/or tracking is desired. By way of example, in certain embodiments food-safe yeast cells containing one or more unique identifier sequences as described herein as part of one or more stably introduced artificial chromosome(s) may be added to or mixed with one or more food products or food ingredients to provide for identification and/or traceability thereof.

Methods for Identification and/or Providing Traceability

In certain embodiments, methods for identification and/or providing traceability of a biological material or biological entity are provided herein. Such methods may utilize a unique identifier sequence to achieve such identification and/or traceability. Typically, a biological entity of interest, such as an agriculture crop (for example, spinach), may be genetically modified to incorporate a unique sequence identifier in its genome. By way of non-limiting and illustrative example, a cell of a spinach plant may be genetically modified to incorporate a cassette, comprising a unique identifier sequence flanked by one or more primer annealing sequences for later amplification and/or sequencing of the unique identifier sequence, into the genome of the spinach cell at an intergenic or other innocuous site of the genome. The sequence of the unique identifier sequence may be known, or may be from a randomized pool and subsequently determined following integration, and may be input and recorded in a database or registry. The cell may then be used to grow/propagate one or more spinach crops, and relevant identification and/or tracking information for the spinach crops (such as source-of-origin, batch/lot information, grower/produced, location, date, vendor, and/or any other supply chain information of interest) may be recorded in the database or registry in association with the corresponding unique identifier sequence. The database entry may, optionally, be updated as supply chain events progress (i.e. harvesting, shipping to a vendor, sale, etc. . . . ). The spinach crop may be used to produce a biological material, such as a bag of spinach or a salad for sale at a grocery store. In the event or suspicion of a contamination or food-borne illness, a sample of a suspect spinach or salad may be obtained, genomic DNA obtained therefrom, and the genomic DNA may be analyzed to determine whether or not a unique identifier sequence is present (i.e. whether or not the spinach is a spinach tracked by the present system) and, if so, the unique identifier sequence may be sequenced to determine the nucleotide sequence, and this nucleotide sequence may be used to provide a query of the database or registry so as to retrieve the relevant database entry providing the identification and/or tracking information so as to facilitate recall of the contaminated spinach or salad. As will be understood, the above spinach example is provided for illustrative purposes, and methods as described herein may be used to provide for a wide variety of identification and/or traceability options for a wide variety of biological entities and/or biological materials in a wide variety of applications.

In an embodiment, there is provided herein a method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the biological material.

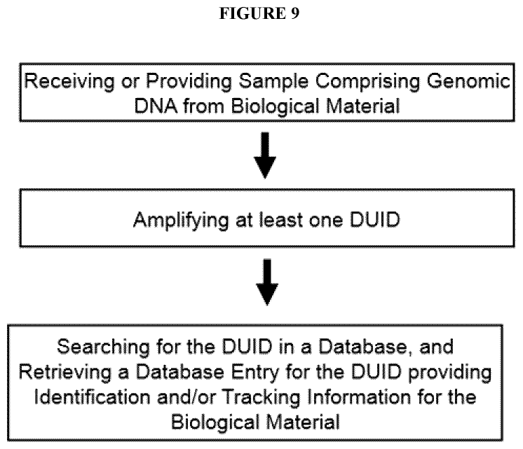

A flow chart depicting an embodiment of such a method is shown in FIG. 9.

As will be understood, the biological material may comprise generally any suitable biological material of interest. The biological material may comprise or consist of a material comprising or consisting of a biological entity, or may comprise or consist of a material made or derived from a biological entity, or any other suitable material of interest which comprises genomic nucleic acid (i.e. genomic DNA) from a biological entity. In certain embodiments, the biological material may comprise or consist of a plant-based material, a fungus-based material, an animal-based material, a virus-based material, or a bacterial-based material. By way of example, in certain embodiments a biological material may comprise or consist of a food or beverage comprising or consisting of or made from a plant or other biological entity, where the food or beverage comprises genomic DNA from the biological entity. In certain embodiments, the biological material may comprise or consist of lettuce, spinach, or other leafy green, or a food product comprising or consisting of or made therefrom, for example.

In certain embodiments of the methods described herein, a sample comprising genomic nucleic acid (i.e. genomic DNA where the biological entity has a DNA-based genome) from a biological material of interest (for example, a biological material for which identification is desired) may be received or provided. The sample may be received or provided in purified or partially purified form such that the genomic DNA may be readily used, or may be provided substantially as-is (i.e. as a sample of the food product) or as another crude or precursor form, which may be subjected to one or more processing or purification steps such that the genomic DNA contained therein may be readily used in subsequent steps. In certain embodiments, it is contemplated that any suitable standard technique for genomic nucleic acid purification and/or isolation may be used for sample preparation.

In certain embodiments, and by way of example, one or more steps of nucleic acid (e.g. genome) isolation, purification, and/or extraction may be performed as part of sample preparation for subsequent steps. DNA isolation or extraction may include, for example, one or more steps for obtaining DNA from a sample. In certain embodiments, DNA isolation or extraction may include breaking open (e.g. lysing) the cells (for example, by physical step(s), sonication, or chemical treatment); removing membrane using a detergent; optionally, removing proteins with a protease; and precipitating DNA using alcohol (such as ethanol (cold) or isopropanol). A DNA pellet may thus be obtained by centrifugation. In certain embodiments, DNAse enzymes may be hindered by using a chelating agent as will be recognized by the skilled person. In certain embodiments, cellular and histone proteins may be removed using protease, or precipitating with sodium or ammonium acetate, or by phenol-chloroform extraction prior to DNA precipitation. The skilled person having regard to the teachings herein will recognize that a wide variety of techniques will be available for sample preparation and/or for isolating, purification, and/or extracting genomic nucleic acid, where desirable.

In certain embodiments of the methods as described herein, a unique identifier sequence (referred to herein as a DNA unique identifier sequence, DUID, for convenience, although it will be understood that in certain examples, such as where the biological entity has an RNA-based genome, the unique identifier sequence may be RNA rather than DNA) inserted or integrated within the genome of the biological entity/biological material may, optionally, be amplified.

In certain embodiments, integration within the genome may include integration within a native chromosome. In certain embodiments, integration within the genome may include stably introducing an artificial chromosome into the genome, the artificial chromosome having centromeric sequence and being heritable along with the native genomic material. Example 2 below describes an example using artificial chromosomes in yeast, for example.

Such amplification may be performed using generally any suitable amplification technique known to the person of skill in the art having regard to the teachings herein, such as by polymerase chain reaction (PCR). In certain embodiments, as described in further detail herein, the unique identifier sequence to be amplified may be accompanied in the genome by primer annealing sequences for amplification and/or sequencing. In certain embodiments, primer annealing sequences may be selected and arranged so as to allow for amplification by nested PCR to reduce likelihood of unintended or off-target amplification, as described in further detail herein.

In certain embodiments, a PCR-based approach may be used for amplification. PCR amplification may involve forward and reverse primers, where the primers may be complementary (or substantially complementary) to regions 5′ and 3′ to the ends of the nucleic acid sequence of interest to be amplified. Forward and reverse primers to specific primer annealing sequences may be produced by any suitable approach known to the skilled person. Examples of such approaches may be found, for example, in Dieffenbach C W, Dveksler G S. 1995. PCR primer: a laboratory manual, New York, N.Y.: Cold Spring Harbor Laboratory Press; New England Biolabs Inc., 2007-08 Catalog & Technical Reference, herein incorporated by reference. In certain embodiments, for reading bio-information, PCR primers may comprise a plurality of sets of forward and reverse primers that may operate independently from one another. In certain embodiments, identity of some primers may be provided or distributed while access to others may be controlled, such that different parties may be able to readily access different regions and/or nucleic acid sequence information as desired.

In certain embodiments of the methods as described herein, a unique identifier sequence, such as a DNA unique identifier sequence (DUID), may comprise any suitable nucleic acid sequence which has been exogenously introduced into the genome of a biological entity for the purposes of identification. Generally, a unique identifier sequence may be either DNA or RNA such that it matches the genome type (DNA or RNA) of the biological entity. As will be understood, the genome of many biological entities, such as plants for example, is double-stranded, and so the unique identifier sequence will typically be found in the genome in double-stranded form. Thus, it will be understood that in certain embodiments, references herein to the unique identifier sequence (such as when describing sequencing of the identifier sequence, for example) may be understood as referencing either strand of the double-stranded construct, or both, as desired or appropriate.

In certain embodiments, the unique identifier sequence may be incorporated into a cassette or other such construct containing one or more functional elements in addition to the unique identifier sequence. In certain embodiments, the cassette may comprise the unique identifier sequence flanked by one or more primer annealing sequences for PCR amplification of the DNA unique identifier sequence, sequencing of the DNA unique identifier sequence, or both. As will be understood, a primer annealing sequence may refer to a pre-determined sequence or region of nucleic acid having a known nucleotide sequence such that one or more primers may be designed or selected for annealing to such primer annealing sequence so as to prime polymerization by a polymerase, for example. Typically, the primer annealing sequences will be selected such that they are unique within the genome of the biological entity of interest so as to reduce or eliminate unintended or off-target amplification. In certain embodiments, the unique identifier sequence may be a known pre-determined sequence selected for a particular application, or may be a random sequence derived from a randomized pool of nucleic acid sequences which may subsequently be determined and recorded in a database as described in detail herein, for example. In certain embodiments, the unique identifier sequence, or the cassette comprising the unique identifier sequence, may have a size of up to about 1500 nt in length; up to about 1000 nt in length; about 200 nt to about 600 nt in length; about 200 nt to about 400 nt in length; or about 400 nt to about 600 nt in length; or any size or subrange spanning between any two of these sizes. As will be understood, longer unique identifier sequences may allow for more unique sequences within a pool, and may allow for reduced risk of duplication. Further, in embodiments where encoding or encrypting of identification information within the unique identifier sequence is desired, longer lengths may allow for relatively more information to be stored and/or more elaborate encryption or encoding schemes to be used, for example. That said, by maintaining a reasonable length such as those referred to herein, a more reliable and/or rapid amplification and/or sequencing may be performed, and/or costs may be relatively reduced.

In certain embodiments, the unique identifier sequence may comprise a sequence of up to about 1500 nt in length; up to about 1000 nt in length; about 200 nt to about 600 nt in length; about 200 nt to about 400 nt in length; or about 400 nt to about 600 nt in length. In certain embodiments, the unique identifier sequence may be relatively short, such as for example about 20 bp in length. As will be understood, in certain embodiments size of the unique identifier sequence may be selected to suit the particular implementation and the desired parameters thereof. In certain embodiments, the unique identifier sequence may have a size of about 20 nt to about 1500 nt, or any size therebetween or any subrange contained therein.

In certain embodiments, the unique identifier sequence may be obtained from a pool at random and may, optionally, be screened for acceptability (e.g. screened for uniqueness, screened to avoid undesirable sequence motifs), or may be rationally designed (e.g. designed for uniqueness, designed to avoid undesirable sequence motifs), for example.

In certain embodiments, the DNA unique identifier sequence may be flanked by one or more primer annealing sequences for PCR amplification of the DNA unique identifier sequence, sequencing of the DNA unique identifier sequence, or both.

In certain embodiments, it is contemplated that the unique identifier sequence may be provided in a cassette or otherwise introduced or inserted into the genomic nucleic acid such that it is flanked by one or more primer annealing sequences for PCR amplification of the DNA unique identifier sequence, sequencing of the DNA unique identifier sequence, or both. Examples of suitable cassettes and configurations are described in further detail herein. In certain embodiments, the cassette may be incorporated into a plasmid, vector, or other such carrier suitable for use in inserting/incorporating/integrating the cassette into the genome of a biological entity.

As will be understood, any suitable genetic modification technique known to the person of skill in the art having regard to the teachings herein may be used for introducing/inserting/incorporating/integrating the unique identifier sequence, or cassette/vector comprising the unique identifier sequence, into the genome of the biological entity. As will also be understood, the genetic modification technique may be selected based on the unique identifier sequence or cassette/vector being used, and based on the particular biological entity being modified. Techniques for genome modification of a wide variety of biological entities, including plants, animals, fungus, bacteria, and viruses, are well-known and may be readily adapted for exogenously introducing a unique identifier sequence as described herein.

By way of example, the skilled person having regard to the teachings herein will be aware of vectors for incorporating DNA into an organism, which may be designed according to known principles of molecular biology. Such vectors may, for example, be designed to stably introduce a DNA sequence of interest into the genome of an organism. In certain embodiments, vectors may be of viral origin or derived therefrom, for example. Where the organism is a plant, it is contemplated that, for example, Agrobacterium tumefaciens-mediated incorporation of DNA of interest may be used for introduction into the plant. The skilled person having regard to the teachings herein will be aware of several other transformation methods, such as ballistic or particle gun methods, among others, which may be adapted as desired or as suitable based on the particular application of interest. In certain embodiments, a gene delivery system may be used based on genetic engineering principles such that sequence of interest may be introduced or inserted into the genome of the host organism. By way of example, in an embodiment, a transposon system may be used for insertion into the genome of a host, which may be a microorganism, animal cell, or plant cell, for example (Insect Molecular Biology (2007), 16(1), 37-47, Plant Physiology Preview. 2007, DOI: 10.1104/pp. 107.111427, the American Society of Plant Biologists; research on production of lactoferrin from transformed silkworms and functionality thereof, the Ministry of Agriculture and Forestry, 2005). In certain embodiments, any suitable method in the field of molecular biology and/or genetic engineering may be used which is able to insert one or more DNA fragments or components of interest into a genome of a host (see, for example, Transgenic Plants Methods and Protocols., Methods in Molecular Biology 2019, Editors: Kumar, Sandeep, Barone, Pierluigi, Smith, Michelle, ISBN 978-1-4939-8778-8, herein incorporated by reference in its entirety).

In certain embodiments, where identification of a biological material or biological entity comprising a unique identifier sequence is desired, the sequence of the unique identifier sequence may be determined by sequencing. As will be understood, the unique identifier sequence may be sequenced by generally any suitable sequencing technique known to the person of skill in the art having regard to the teachings herein. In certain embodiments, the sequencing may be assisted by the inclusion or use of a sequencing primer annealing sequence associated with the unique identifier sequence within the genomic nucleic acid. Examples of such sequencing primer anneal sequence, which may be incorporated into a cassette comprising the unique identifier sequence, for example, are described in detail herein.

In certain embodiments, sequencing may be performed using any suitable sequencing technique known to the person of skill in the art having regard to the teachings herein, which may be selected based on the particular application and/or configuration being used. In certain embodiments, sequencing may be performed by any suitable sequencing method for determining the order of nucleotide bases in a molecule of DNA (or RNA). Examples of sequencing methods may include, for example, Maxam-Gilbert sequencing, chain termination methods, dye-terminator sequencing, automated DNA sequencing, in vitro cloning amplification, parallelized sequencing by synthesis, sequencing by ligation, Sanger sequencing such as microfluidic Sanger sequencing and sequencing by hybridization, for example.

In certain embodiments, once the sequence of a unique identifier sequencing of a biological material is determined, the sequence may be used to provide a query for searching in a database (also referred to herein as a registry) containing a collection of unique identifier sequences paired or otherwise associated with relevant identification and/or tracking information. If a matching database entry is found, the database entry may be retrieved so as to provide identification and/or tracking information for the biological material of interest. In such manner, relevant identification and/or tracking information for the biological material may be determined, and may be used, for example, to inform an event such as, for example, a food recall or other action.

In another embodiment, there is provided herein a method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and comparing the sequence of the DNA unique identifier sequence with a database to confirm that the DNA unique identifier sequence is not already used in the database;
providing an indication of acceptability to produce a biological material from the biological entity, the biological material comprising genomic DNA from the biological entity; and
inputting the sequence of the at least one DNA unique identifier sequence into a database entry of the database, and associating the DNA unique identifier sequence with identification and/or tracking information for the biological material;

thereby providing traceability of the biological material by reading the DNA unique identifier sequence in the biological material and retrieving the corresponding database entry providing the identification and/or tracking information for the biological material.

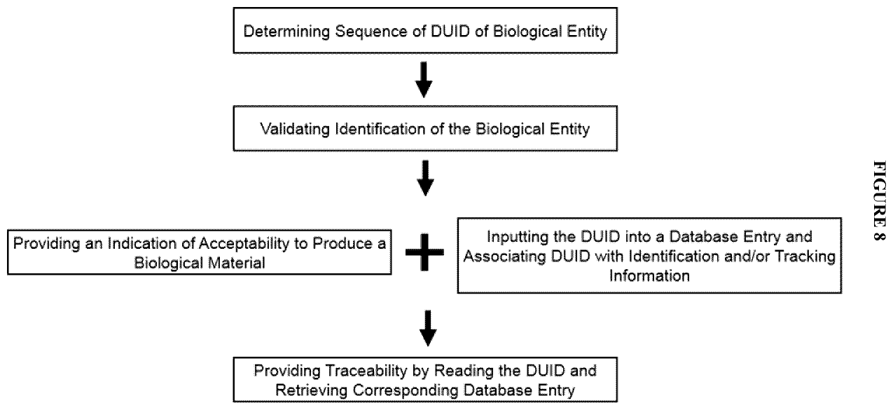

A flow chart depicting an embodiment of such a method is shown in FIG. 8.

As will be understood, the biological entity may comprise generally any suitable biological entity of interest. The biological entity may comprise or consist of a cell (i.e. a plant cell, fungal cell, animal cell, or bacterial cell), or a seed or tissue comprising one or more cells, or a virus, or an organism such as a plant, animal, or fungus, or any portion thereof. In certain embodiments, the biological entity may comprise a plant cell, a fungal cell, an animal cell, a virus, or a bacterial cell. Where the biological entity is to be genetically modified to incorporate a unique identifier sequence, the biological entity may typically comprise a cell or virus which may be propagated following the genetic modification to produce more biological entities each comprising the inserted unique identifier sequence.

In certain embodiments, the step of validating may be performed to verify the presence of the unique identifier sequence within the genomic DNA of the biological entity, and/or to determine the sequence thereof, and/or to determine if the unique identifier sequence is not already used in the database (i.e. is a new sequence which has not already previously been associated with a database entry). If validation is successful (i.e. the unique identifier sequence is properly inserted and unique to the database), then in certain embodiments a database entry for the unique identifier sequence may be created in the database (which may be associated with relevant identification and/or tracking information, and may optionally be updated on an ongoing basis), and an indication of acceptability to produce a biological material from the biological entity may be provided to an interested party such as a grower, farmer, or other agriculture entity who may then produce or grow the biological material.

In such manner, traceability of the biological material may be provided by reading (i.e. sequencing) the unique identifier sequence of the biological material, which may be used to retrieve the corresponding database entry to obtain the identification and/or tracking information.

In certain embodiments, the methods described herein may further comprise inserting at least one DNA unique identifier sequence within the genomic DNA of a biological entity, or modifying a pre-existing identifier sequence within the genomic DNA of a biological entity by gene editing to create a DNA unique identifier sequence within the genomic DNA of the biological entity, thereby providing identification thereof.

In yet another embodiment, the methods described herein may further comprise providing the at least one DNA unique identifier sequence for the insertion within the genomic DNA of the biological entity. In certain embodiments, the DNA unique identifier sequence may be provided as a randomized pool of sequences as further described herein.

As will be understood, it is contemplated that in certain embodiments methods as described herein may utilize a single unique identifier sequence, or may use two or more identifier sequences incorporated into the genome in order to provide for identification and/or traceability.

In certain embodiments, the unique identifier sequence may be from a randomized pool of unique identifier sequences. The identity of the inserted unique identifier sequence may not be determined until the insertion (i.e. transformation or genetic modification) has been achieved. In such manner, it is contemplated that interested parties may be provided with a randomized pool of unique identifier sequences, and may perform genetic modification of a biological entity of interest such that one, two, or more unique identifier sequence(s) become inserted in the genome. Following the genetic modification process, the inserted unique identifier sequence(s) may be sequenced to determine the nucleotide sequence of the inserted unique identifier sequence(s). Given that the typical length of a unique identifier sequence may typically be selected to be sufficiently long so as to provide a vast number of different sequences within the randomized pool, the statistical likelihood of two different parties inserting the same unique identifier sequence may be extremely low. Accordingly, in such manner, it is contemplated that in certain embodiments many different parties seeking to benefit from identification and/or traceability of methods as described herein may all be provided with a sample from the same a similar randomized pool of sequences for insertion in their biological entities of interest. In such manner, it is contemplated that processes may be streamlined and/or costs may be reduced in certain embodiments.

In yet another embodiment of methods as described herein, reading the DNA unique identifier sequence in the biological material and retrieving the corresponding database entry may comprise:
receiving or providing a sample comprising genomic DNA from the biological material;
amplifying the at least one DNA unique identifier sequence within the genomic DNA from the biological material and sequencing the DNA unique identifier sequence; and
comparing the DNA unique identifier sequence to the database and retrieving the database entry corresponding with the DNA unique identifier sequence, the database entry providing identification and/or tracking information for the biological material.

In certain embodiments, it is contemplated that the unique identifier sequence(s) may be inserted into the genome of the biological entity at a site which is substantially innocuous (i.e. may not substantially affect gene expression or phenotype). For example, in certain embodiments, it is contemplated that the unique identifier sequence(s) may be inserted at one or more intergenic region(s) of the genomic DNA.

In certain embodiments, the identification and/or tracking information provided in the database or registry may comprise supply chain information for the biological material. In certain embodiments, the identification and/or tracking information of the database may comprise source-of-origin information for the biological material. In certain embodiments, the identification and/or tracking information of the database may comprise grower, region, batch, lot, date, or other relevant supply chain information, or any combinations thereof. The person of skill in the art having regard to the teachings herein will be aware of a variety of identification and/or tracking information that may be included in the database, and may be selected as desired or to suit a particular application. In certain embodiments, existing supply chain tracking features, such as a barcode or lot or batch number, may be included in the database, for example.

In certain embodiments, information such as geographic region, dates, buyers, farmers, lots, sub-lots, harvests, batches, other DUID-enabled products, organisms, contractual obligations, certifications, neighbouring industry and businesses, sensor data, weather data, or any combinations thereof, may be included/stored in the database.

In another embodiment, there is provided herein a method of identifying a biological material, the method comprising:
receiving at a computing device a DNA-unique identifier sequence (DUID) extracted from a known biological material;
searching at the computing device a DUID database storing a plurality of DUIDs in association with respective biological material information for a match to the received DUID;
if the search of the DUID database fails to provide a match to the received DUID, storing in the DUID database the received DUID in association with biological material information associated with the known biological material;
subsequent to storing the received DUID and with information associated with the known biological material in the DUID database, receiving at the computing device a query DUID extracted from an unknown biological material;
searching at the computing device the DUID database for a match to the received query DUID; and
if the search of the DUID provides a match to the received query DUID, returning in response to the received query DUID the biological information stored in association with the DUID matching the query DUID.

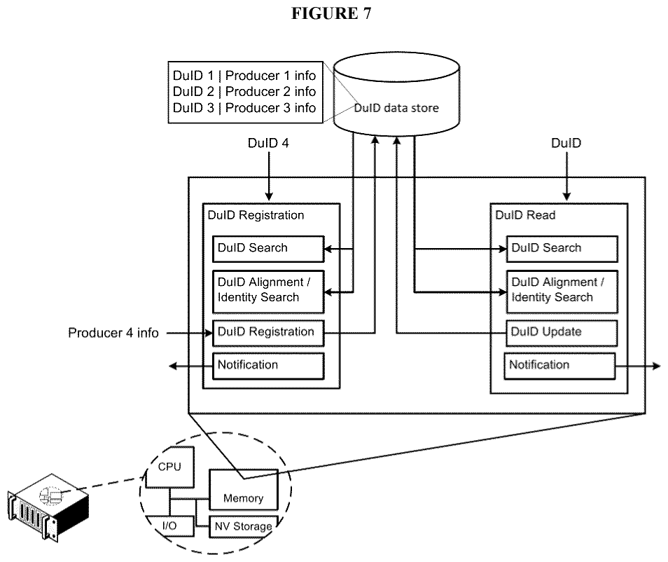

A flow chart depicting an embodiment of such a method is shown in FIG. 7. In this Figure, a DNA-unique identifier sequence (DUID—DuID 4 in the depicted example) is extracted (i.e. read, determined, or sequenced) from a known biological material and provided to a computing device. The computing device is used for searching a DUID database (i.e. a DuID data store) storing a plurality of DUIDs in association with respective biological material information, for a match to the received DUID 4. If the search of the DUID database fails to provide a match to the received DUID, the received DUID (DuID 4) is stored in the DUID database in association with biological material information (i.e. Producer 4 info) associated with the known biological material, thus providing registration of the DUID and the biological material in the database. An interested party may then be provided with a notification of successful registration, and approved to proceed with propagating the biological entity/material to produce a biological material such as a food product. Subsequent to storing the received DUID and with information associated with the known biological material in the DUID database, a query DUID extracted (i.e. read, for example by sequencing) from an unknown biological material (i.e. a biological material of interest, such as a food product suspected of contamination) may be received at the computing device, and a search of the DUID database may be performed for a match to the received query DUID. If the search of the DUID database provides a match to the received query DUID, the biological information stored in association with the DUID matching the query DUID may be returned in response to the received query DUID, thus providing tracking and/or identification information for the biological material, which may be used to take a response such as, for example, a food recall.

In another embodiment, searching the DUID database for a match to the received DUID may comprise:
searching the DUID database for an exact match to the received DUID; and
if an exact match is not found, performing an alignment/identity search for DUIDs stored in the DUID database that are a close match to the received DUID.

In still another embodiment, searching the DUID database for a match to the query DUID may comprise:
searching the DUID database for an exact match to the query DUID; and
if an exact match is not found, performing an alignment/identity search for DUIDs stored in the DUID database that are a close match to the query DUID.

As will be understood, since nucleic acid sequence is being used, there may be a possibility for sequence mutation of the unique identifier sequence during propagation and/or amplification and/or sequencing errors may occur. Accordingly, in certain embodiments, such an alignment/identity search may be performed to identify whether an entry for a close or highly similar match may exist. A variety of sequence comparison algorithms exist for performing such alignment/identity/similarity assessment (see, for example, BLAST tools available from the NCBI), and the skilled person having regard to the teachings herein will be able to select or adapt an appropriate algorithm as desired to suit a particular application.

In yet another embodiment, the methods described herein may further comprise:
if the search provides a close match to the query DUID, storing the query DUID in association with the DUID that is a close match to the query DUID.

In such manner, the database may be updated where, for example, sequence mutation is identified, for example.

In another embodiment, there is provided herein a computing system for identifying a biological material, the system comprising:
a processing unit capable of executing instructions; and
a memory unit storing instructions, which when executed by the processing unit configure the computing system to perform any of the method or methods as described herein.

In another embodiment, there is provided herein a computer readable memory, having instructions stored thereon, which when executed by a processing unit of a computing system configure the system to perform any of the method or methods described herein.

In another embodiment, there is provided herein a method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
decoding or decrypting identification and/or tracking information for the biological material stored in the DNA unique identifier sequence.

Such method embodiments may be similar to those described herein utilizing a database or registry, with the exception that rather than storing identification and/or tracking information in the database, the information may instead be encoded (encrypted or not) within the unique identifier sequence itself. Approaches for storing information in nucleic acid sequence are known in the field, and may typically involve using A, T, G, C nucleotides similarly to 0 and 1 bits in digital data storage. An example of approaches for storing/encoding/encrypting information may be found, for example, in Clelland, C., Risca, V. & Bancroft, C. Hiding messages in DNA microdots. Nature 399, 533-534 (1999) doi:10.1038/21092 (herein incorporated by reference).

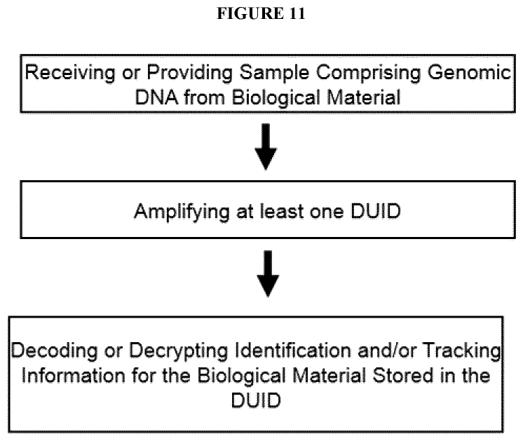

A flow chart depicting an embodiment of such a method is shown in FIG. 11.

In certain embodiments, it is contemplated that the unique identifier sequence may be used to encode a key, and it is the key which is stored in the database in association with the tracking and/or identification information. Thus, it is will be understood that references herein to storing the DUID in the database, and searching the database for the DUID, may be considered as encompassing both direct (i.e. storing and searching for the primary nucleic acid sequence of the unique identifier sequence itself), and indirect (i.e. obtaining a key from the primary nucleic acid sequence of the unique identifier sequence, and using the key to store in the database and to search the database) options. The skilled person having regard to the teachings herein will be aware of a variety of combinations which may be used, all of which are intended to be encompassed herein.

In another embodiment, there is provided herein a method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and decoding or decrypting identification and/or tracking information stored in the DNA unique identifier sequence to verify the DNA unique identifier sequence; and
providing an indication of acceptability to produce a biological material from the biological entity, the biological material comprising genomic DNA from the biological entity;

thereby providing traceability of the biological material by reading the DNA unique identifier sequence in the biological material and decoding or decrypting information stored in the DNA unique identifier sequence, providing identification and/or tracking information for the biological material.

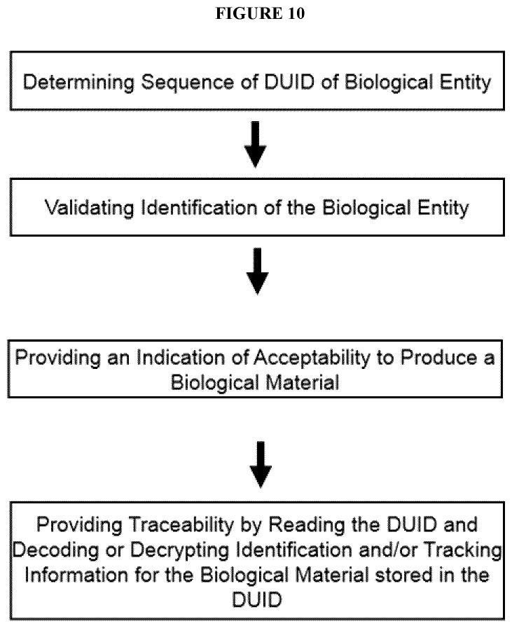

A flow chart depicting an embodiment of such a method is shown in FIG. 10.

Such method embodiments may be similar to those described herein utilizing a database or registry, with the exception that rather than storing identification and/or tracking information in the database, the information may instead be encoded (encrypted or not) within the unique identifier sequence itself. Approaches for storing information in nucleic acid sequence are known in the field, and may typically involve using A, T, G, C nucleotides similarly to 0 and 1 bits in digital data storage. An example of approaches for storing/encoding/encrypting information may be found, for example, in Clelland, C., Risca, V. & Bancroft, C. Hiding messages in DNA microdots. Nature 399, 533-534 (1999) doi:10.1038/21092 (herein incorporated by reference).

In still another embodiment, there is provided herein a method of identifying a biological material, the method comprising:
receiving at a computing device a DNA-unique identifier sequence (DUID) extracted from an unknown biological material; and
decoding or decrypting identification and/or tracking information for the unknown biological material stored in the DNA unique identifier sequence.

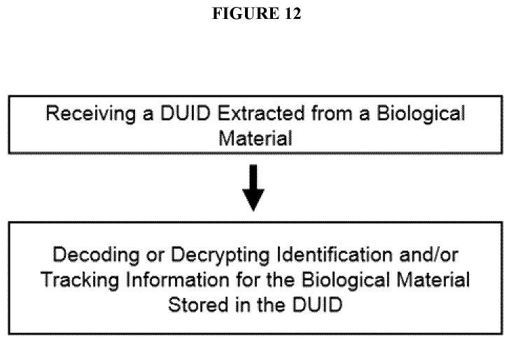

A flow chart depicting an embodiment of such a method is shown in FIG. 12.

In yet another embodiment, there is provided herein a method for providing traceability of biological material, said method comprising:
inserting at least one DNA unique identifier sequence within the genomic DNA of a biological entity for use in preparing the biological material.

In another embodiment of the above method, the DNA unique identifier sequence may be inserted as any of the cassette or cassettes as described herein.

In another embodiment of any of the above method or methods, the method may further comprise a step of determining the sequence of the least one DNA unique identifier sequence within the genomic DNA of the biological entity.

In another embodiment of any of the above method or methods, the method may further comprise a step of validating identification of the biological entity by: verifying presence of the DNA unique identifier sequence in the genomic DNA; and comparing the sequence of the DNA unique identifier sequence with a database to confirm that the DNA unique identifier sequence is not already used in the database.

In still another embodiment of any of the above method or methods, the method may further comprise a step of:
producing the biological material from the biological entity, the biological material comprising genomic DNA from the biological entity; and/or
providing an indication of acceptability to produce the biological material from the biological entity, the biological material comprising genomic DNA from the biological entity.

In still another embodiment of any of the above method or methods, the method may further comprise a step of inputting the sequence of the at least one DNA unique identifier sequence into a database entry, and associating the DNA unique identifier sequence with identification and/or tracking information for the biological entity and/or biological material.

In yet another embodiment of any of the above method or methods, the method may further comprise a step of:
providing traceability of the biological entity and/or biological material by reading the DNA unique identifier sequence in the biological entity and/or biological material and retrieving the corresponding database entry providing the identification and/or tracking information for the biological entity and/or biological material.

Oligonucleotide Constructs, Cassettes, Plasmids, Vectors, Cells, and Kits

In another embodiment, there is provided herein a cassette comprising a unique identifier sequence, the unique identifier sequence flanked by at least one 5′ primer annealing sequence and at least one 3′ primer annealing sequence for amplification of the DNA unique identifier sequence, sequencing of the DNA unique identifier sequence, or both.

As will be understood, in certain embodiments, such cassettes may be for use in any of the method or methods as described herein.

In certain embodiments of the cassette, the DNA unique identifier sequence may be flanked by two 5′ primer annealing sequences and two 3′ primer annealing sequences to allow for amplification of the DNA unique identifier sequence by nested PCR. In certain embodiments, a nested design may be used to improve recall fidelity, for example. In still a further embodiment of the cassette, the two 5′ primer annealing sequences may be partially overlapping; the two 3′ primer annealing sequences may be partially overlapping; or both. In still a further embodiment of the cassette, the cassette may further comprise a sequencing primer annealing sequence located 5′ to the DNA unique identifier sequence for sequencing of the DNA unique identifier sequence. In yet a further embodiment of the cassette, the sequencing primer annealing sequence may be positioned between two 5′ primer annealing sequences. In a further embodiment of the cassette, the sequencing primer annealing sequence may at least partially overlap with one or both of the two 5′ primer annealing sequences. In yet a further embodiment of the cassette, the two 5′ primer annealing sequences may be partially overlapping, and at least a portion of the sequencing primer annealing sequence may be positioned at the overlap. In a further embodiment of the cassette, the cassette sequence may be up to about 1500 nt in length; up to about 1000 nt in length; about 200 nt to about 600 nt in length; about 200 nt to about 400 nt in length; or about 400 nt to about 600 nt in length.

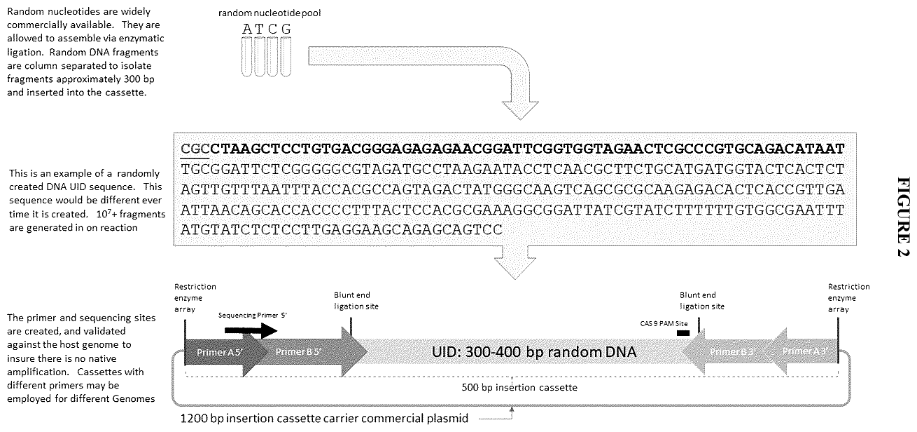

An embodiment of a cassette as described herein, and a example of a process for the production thereof, is shown in FIG. 2, in which a cassette may be produced using a pool of oligonucleotides of randomized sequence. Randomized pools of oligonucleotides may be commercially obtained, or synthesized as desired. They may be assembled via enzymatic polymerization or ligation, or chemically synthesized, for example. Random oligonucleotide fragments may be purified, for example by column separation, to isolate fragments of approximately the same or similar size (for example, about 300 nt-400 nt in size in the depicted example), and may be inserted into the cassettes. A pool of cassettes containing a vast variety of different unique identifier sequences (i.e. about 10⁷ in some examples) may be produced. The cassette may comprise primer annealing sequences (i.e. primer binding sites) and at least one sequencing primer annealing sequence (i.e. sequencing primer binding site), in a suitable arrangement so as to allow for amplification and/or sequencing of the DUID, such as the configuration as shown in FIG. 2. Primer and sequencing sites may be validated against the host genome to verify that there is no native amplification. Cassettes with different primers may be employed for different organisms or for different genomes, if desired. The cassette may comprise restriction enzyme array sites, and may be provided in the form of an insertion cassette carrier plasmid or vector, for example. In certain embodiments, the cassette may be about 500 bp in length, and may be provided within a plasmid or carrier vector of about 1200 bp in size, for example.

As will be understood, a primer annealing sequence of a cassette may refer to a pre-determined sequence or region of nucleic acid having a known nucleotide sequence such that one or more primers may be designed or selected for annealing to such primer annealing sequence so as to prime polymerization by a polymerase, for example. Primer annealing sequence may be used for amplification of the unique identifier sequence, sequencing of the unique identifier sequence, or both.

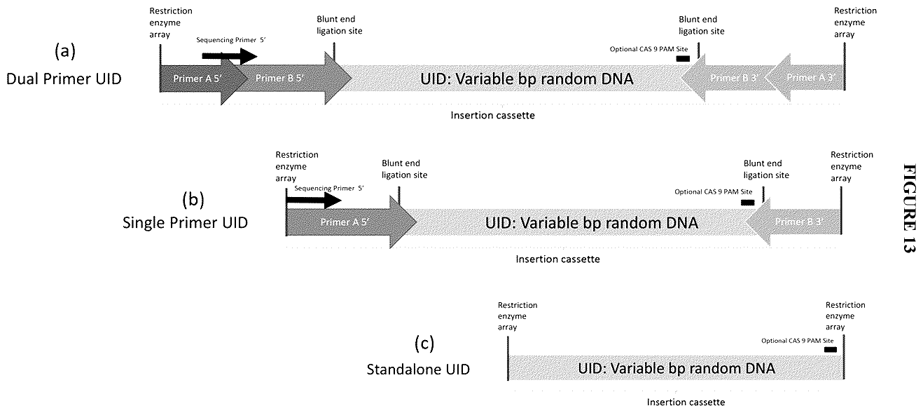

FIG. 13 shows additional examples of cassette designs as described herein including a UID (unique identifier) sequence. FIGS. 13(a) shows a dual primer design, 13(b) shows a single primer design, and 13(c) shows a standalone design. In the dual primer insertion cassette design of FIG. 13(a), the depicted embodiment includes a restriction enzyme array, a 5′ “Primer A” region and a 5′ “Primer B” region (where 5′ sequencing primer may anneal at a region spanning between “Primer A” and “Primer B” regions), followed by a blunt end ligation site. Next, a UID region (e.g. variable bp random DNA, or another identifier sequence) is provided, and a CAS 9 PAM site may, optionally, be provided as shown. A blunt end ligation site follows, and then a 3′ “Primer B” region and a 3′ “Primer A” region is provided, followed by a restriction enzyme array. In the single primer insertion cassette design of FIG. 13(b), the depicted embodiment includes a restriction enzyme array, a 5′ “Primer A” region (where 5′ sequencing primer may anneal), followed by a blunt end ligation site. Next, a UID region (e.g. variable bp random DNA, or another identifier sequence) is provided, and a CAS 9 PAM site may, optionally, be provided as shown. A blunt end ligation site follows, and then a 3′ “Primer B” region is provided, followed by a restriction enzyme array. In FIG. 13(c) an embodiment of a standalone insertion cassette design is depicted, which includes a restriction enzyme array, a UID region (e.g. variable bp random DNA, or another identifier sequence), a CAS 9 PAM site may, optionally, be provided, and a restriction enzyme array, as shown.

As shown in FIG. 13, a variety of different cassette designs are contemplated. Cassettes may vary, for example, in terms of elements present, in terms of size, and in terms of amplification efficiency. Depending on whether primer pairs are present (see FIGS. 13(A)-(C)), total cassette size may change. For example, as individual primer pairs are eliminated, total cassette size may be reduced (for example, by about 40 bp in certain embodiments). As will be understood, in certain embodiments amplification efficiency for the UID may decrease as a result of primer pair elimination. For example, for a dual primer design, any permutation of the primers may be used for amplification, giving 4 possible variations rather than one as would be found for a single primer pair design. As will also by understood, in certain embodiments, reducing cassette size may provide for a reduction in the potential for unintended effects, for example. In certain embodiments, an optional CAS 9 PAM site may be used to permit for efficient CRISPR-based editing of the UID sequence amongst transformed organism progeny, for example. In certain embodiments in which all primers are eliminated from the cassette design, it is contemplated that a CAS 9 PAM may, optionally, be provided, where the CAS 9 PAM site may, in certain embodiments, permit the standalone cassette to be constructed entirely of host genome DNA, such as when using a DNA digestion/ligation technique, for example. In certain embodiments, the UID sequence may be variable in length. It is contemplated that in certain embodiments, even short UID sequences may be safely used, particularly where a validation step is performed that includes a check for any collisions amongst existing UIDs in the registry and the newly inserted UID, for example.

In still another embodiment of the cassettes described herein, the primer annealing sequences may not be naturally occurring in the genome of a target biological entity. In such manner, unintended and/or off-target amplification and/or sequencing may be reduced or avoided.

In another embodiment, there is provided herein a composition comprising a plurality of any of the cassette or cassettes as described herein, each cassette comprising the same primer annealing sequences, and each cassette comprising a randomized DNA unique identifier sequence. Such compositions may represent an example of a randomized pool of sequences as described herein.

In still another embodiment, there is provided herein a composition comprising a plurality of any of the cassette or cassettes as described herein, each cassette comprising the same primer annealing sequences and the same sequencing primer annealing sequence, and each cassette comprising a randomized DNA unique identifier sequence. Such compositions may represent an example of a randomized pool of sequences as described herein.

In another embodiment, there is provided herein a plasmid, expression vector, or other single or double-stranded oligonucleotide construct comprising any of the oligonucleotide or oligonucleotides as described herein, or any of the cassette or cassettes as described herein.

In another embodiment, there is provided herein a cassette comprising any of the oligonucleotide or oligonucleotides as described herein.

In still another embodiment, there is provided herein a cell or virus comprising any of the oligonucleotide or oligonucleotides as described herein, or any of the cassette or cassettes as described herein, incorporated into the genome of the cell or virus. In another embodiment, there is provided herein a cell or virus comprising a unique identifier sequence incorporated into the genome of the cell or virus. In another embodiment of any of the cells or viruses described herein, the unique identifier sequence may be incorporated into an intergenic region of the genomic nucleic acid of the cell or virus. In still another embodiment of any of the cells or viruses, the cell may be a plant cell, a fungal cell, an animal cell, or a bacterial cell.

In another embodiment, there is provided herein a kit comprising any one or more of:
a DNA unique identifier sequence;
a randomized pool of DNA unique identifier sequences;
any of the oligonucleotide or oligonucleotides as described herein;
any of the cassette or cassettes as described herein;
one or more primers or primer pairs for amplifying and/or sequencing a DNA unique identifier sequence;
a buffer;
a polymerase; or
instructions for performing any of the method or methods as described herein;
or any combinations thereof.

In yet another embodiment, there is provided herein a method for providing traceability of a product of interest, said method comprising:
receiving or providing a sample from the product of interest, the sample comprising genomic DNA from a biological material part of, mixed with, or otherwise associated with the product of interest;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the product of interest.

In another embodiment of the above method, the method may comprise introducing or adding any of the biological material or biological materials or biological entity or biological entities as described herein to the product of interest, the biological material or entity comprising at least one DNA unique identifier sequence as described herein as part of its genomic material.

In yet another embodiment of any of the above method or methods, the identification and/or tracking information of the database entry may comprise supply chain information for the product of interest.

In still another embodiment of any of the above method or methods, the product of interest may comprise food, an agricultural product, a pharmaceutical drug, a retail product, textiles, commodities, chemicals, or another supply chain item.

### EXAMPLE 1

### Exemplary DUID System for Providing Food Traceability

This example describes embodiments of an exemplary food traceability system referred to herein as a DNA unique identifier (DUID) system. This example utilizes the durability and replicative capacity of DNA sequences to safely encode unique identifiers within the nuclear genome of an organism. Encoding identifying information into the DNA of an organism in the presently described manner may provide granularity in traceability across the supply-chain. In particular, the DUID system may have the capacity to:
1. Safely achieve DNA-level population identification without effecting the heritable traits of the target organism;
2. Create logical relationships between the DUID and reference information;
3. Reduce the time taken to trace a product to its source-of-origin from months to about a day;
4. Provide rapid identification of both the product's source-of-origin and its definitive path through the supply chain;
5. Provide valuable information to health care professionals and industry regulators;
6. Foster consumer and industry confidence in the stability, transparency and efficacy of the food supply-chain; and/or
7. Support mechanisms for enforcing membership association obligations, and the intellectual property of food products.

It is contemplated that in certain embodiments the DUID system may be used to significantly augment the surveillance capabilities of food system stakeholders, for example. DUID systems as described herein, in addition to providing traceability, may turn traditional thinking about point of attribution on its head—bottom-up instead of top-down. Such approaches, as described herein, may be particularly desirable given increases in supply-chain consolidation becoming the norm. DUID systems as described herein may provide for virtually guaranteed source-of-origin traceability from generally anywhere throughout the supply-chain, within a about day if desired. Systems may benefit from the replicative and stable cellular properties of an organism, and as a result, marginal costs may approach zero as progeny are created. The financial cost and risk of tampering and/or fraud for traditional tracking systems is quite high, and the legal implications of malicious activity may be significant. DUID systems as described herein may be edited in interesting ways such that a population's progeny maintains portions of the original identifier, for example. The DUID may also be utilized by health care professionals who may want to test human excreta in order to identify recently consumed food, for example.

It is contemplated that aforementioned population-level identification may, optionally, include additional reference to legal agreements. By way of example, it is contemplated that IP owners of a product may purposefully link propagating material to, for example, a particular grower and/or region. Population-level genetic identification in conjunction with traditional whole-chain traceability techniques may enable remarkable levels of control over the movement of product. Consider, for example, a spinach plant variety that has been genetically engineered to be resistant to various pests. Using a DUID system, there may be significantly reduced costs in detection. In addition to cost reduction, the DUID system may play a role as a registry to provide a centralized point of contact for IP tracking, for example.

Many organisms are regulated. For example, a plant variety may be a precursor to a narcotic. Such organisms may, in certain embodiments, benefit from being inextricably associated with an approved legal entity, for example. Accordingly, it is contemplated that such instances may benefit from strategies as described herein.

In an example, consider regulation of cannabis in Canada. The production and distribution of cannabis plants and their propagating material is regulated. It is contemplated that in certain embodiments, licensed cannabis producers may include a DUID into their products, for example, which may be used to assist with regulation. In certain embodiments, such DUID may be helpful for regulation by identifying and/or tracking cannabis, even in complex instances where cannabis is mixed with something else (i.e. in edible products, for example).

In another example, consider a spinach growers association. Membership to the association may be required in order to grow and sell spinach in certain examples. In certain embodiments, it is contemplated that such propagating materials may have been derived from a DUID-ready plant. Random audits may then be done at the retail level to ensure all spinach being sold is accredited, for example.

DUID System:

The DUID system may encompass, for example, product identification, DUID validation, DUID reads, and the subsequent tracking of populations of products. It may also function as a central registry for all DUID data.

In the following examples, the DUID platform may comprise a collection of actors, business services, tasks, events, and systems. Actors may execute or trigger business services and tasks. Systems and business services may be understood in terms of the events that they produce.

Events may be directly linked to the trace state of a food product.

Actors: By way of example, a consumer safety officer (Actor) from the FDA (Actor) may request that the DUID Platform (Actor) attempts to read (Business Service) the DUID from a supplied organic material of interest. Actors are engines of the DUID platform. Actors may be systems, organizations, and/or individuals. They may trigger events and make requests to business services. Actors may also execute tasks. The following list provides some examples of actors; however, this is a non-exhaustive list intended for illustrative purposes:

DUID Platform
DUID Registry
DUID API
Analytical Chemist
Microbiologist
DNA Sequencer

Producer
Botanist
CFO
Traceability Software

Grower
Director of Food Safety
Enterprise Resource Planning System

Packer/Shipper
Truck Driver
Manager
CEO

Retailer
Director of Food Safety
CFO
General Counsel

Government Regulator
Consumer Safety Officer
Management Officer

Insurer
Underwriter
Claims Adjuster

Business Services: By way of example, upon authentication/authorization of the consumer safety officer (Actor) and the successful completion of the read (Business Service), a read (Event) may be logged in the registry (System). Business services may encompass critical processes and tasks, which may ultimately produce an event. These services may be designed to be stateless in that they do not require any particular prior state exist in order for it to be triggered. They may dictate that certain events have occurred in order to complete successfully. In any case, a business service may utilize a system, but most typically includes some human involvement. By way of example, in certain embodiments it should be requested or triggered by an actor. Business services may also be named similarly to the event that they produce—e.g. Validation (business service)→Validated (event).

Systems: by way of example, once the read (Event) has been logged to the registry (system), a stream processor (system) may read the newly created read (event) from the registry and may broadcast it to authenticated/authorized listeners (system). One of the listeners may update a notifications dashboard used by the product's brand owner (actor). Systems on the other hand may be only interacted with by other systems, or otherwise, a client operated by a human. In other words, systems may typically be digital systems. An example of a system within the DUID platform may be an API. The API may expose an interface to authorized actors that operate outside of the platform boundaries. Another example of a system may be the DUID Registry (i.e. database), which may function as the persistent data store for all DUID data. The registry may not be directly exposed to external actors.

Events: By way of example, a read (Business Service) may be requested by a consumer safety officer (Actor) from the FDA (Actor). After authorization/authentication, the business service may result in a successful read (Event). Events may refer to the outcome of business services and systems. Events are typically logged in relation to a DUID. That is, an organism may be identified; validated or read by a business service; and tracked by internal or external systems. The following Table outlines each event, and its relationship to various business services, actors, systems, and tasks in this example.

TABLE 1
Events, and relationships to various business services,
actors, systems, and tasks in this Example.

| | |
|---|---|
| Identified | The identified event may refer to the process through which the cassette is |
| | assembled or edited, inserted into the genome of the organism and subsequently |
| | validated for a range of properties. |
| Validated | The validated event may refer to the outcome of the validating business service. |
| | Validated may indicate the producer has successfully transformed the organism in |
| | question, and they may begin regeneration. |
| Read | The read event may refer to a read of the DUID. It may be necessitated by an |
| | identified event. A read event may be required in order to achieve a confirmed |
| | tracked event. |
| Tracked | The tracked event may refer to all logging activities for the identified organism. |
| | This may include logging the disposition of a product to a supply chain recipient. |
| | Tracked events may be logged as confirmed or unconfirmed. A confirmed tracked |
| | event may require the authenticated read of a DUID in organic material using |
| | common sequencing techniques. Unconfirmed tracked events may be logged using |
| | some sort of tag or barcode external to the organism's DNA - e.g. the identifier |
| | portion of the DUID may be included on barcodes, for example. |

As described, the DUID platform in this example may encompass various actors, business services, events, systems and/or tasks. All of these components may adhere to specific process flow. This section will describes an exemplary flow in detail. The diagrams used to illustrate these processes use the BPMN 2.0 notation (BPMN 2.0—https://www.omg.org/spec/BPMN/2.0/PDF; herein incorporated by reference in its entirety). The diagrams are available in the Figures, which are described in further detail hereinbelow.

Process Overview:

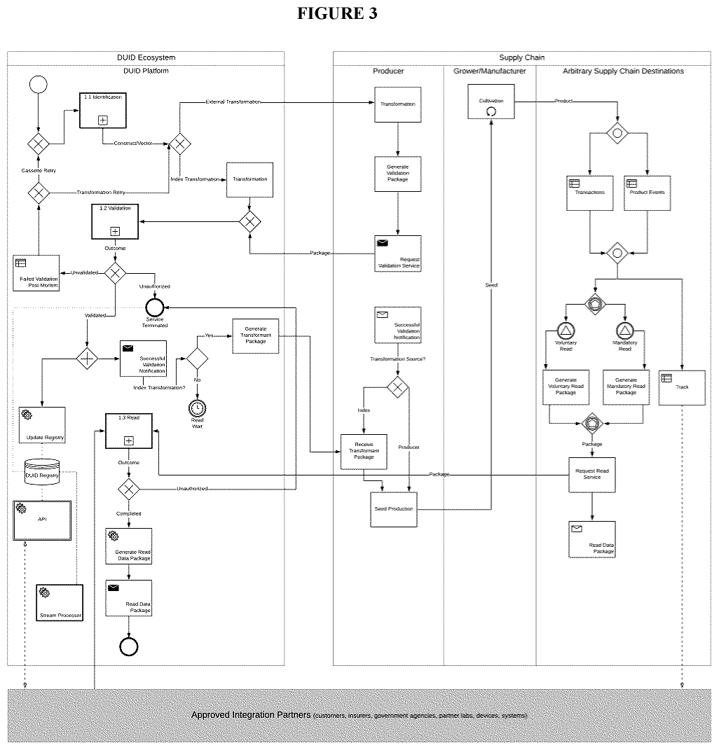

FIG. 3 describes the global view of the exemplary process for the DUID ecosystem of this example.

Process Start:

Before the exemplary process begins, there may be an expectation that the relevant agreements have been put in place regarding the terms of service. This may include know-your-customer (KYC) validations such as proof of ownership, legal entity identification, and payment. In addition to KYC requirements, customers may be able to specify user access roles and other system/account settings via an administrative dashboard.

Primer and Sequencing Site Creation:

This may be an ongoing/running task that may occur independent of the process. The development of DUID primers may depend on customer host organism requirements, or R&D efforts, or both, for example. The existence of usable primers may be used for the identification business service.

Identification:

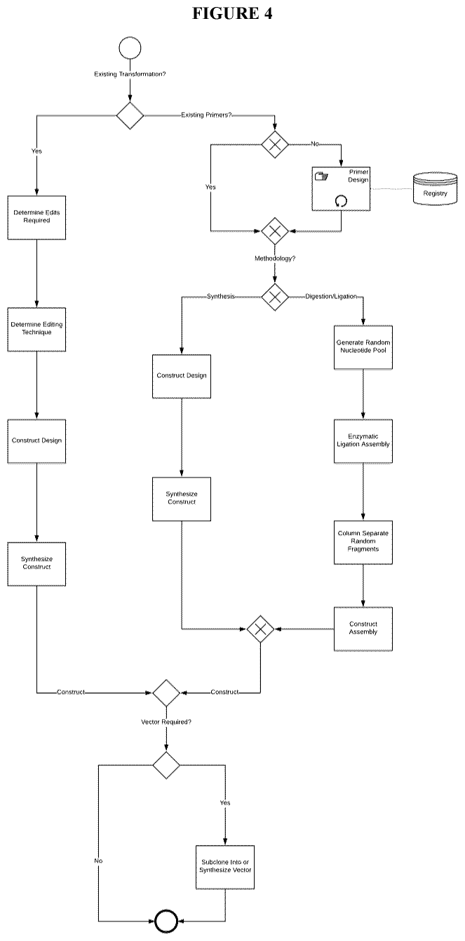

The identification business service may be viewed in detail in FIG. 4. The physical output of this business service may be a DNA sequence-based cassette, which may be used by the producer during organism transformation. There may be two scenarios that may play out within this activity.

First, it is contemplated that if there is an existing cassette, a standard CRISPR and/or related technique may be used to modify portions of the existing identifier. For example, if the existing identifier has been mapped to a geographic region, a few bases may be edited at the end of the sequence. This edit may be mapped to more specific information—e.g. expected transformed state after processing. An identified event may be triggered once this is complete.

If there is no existing cassette, it may be created. See FIG. 2 for details about such a process. As shown in FIG. 2, a cassette may be produced using a pool of oligonucleotides of randomized sequence. Randomized pools of oligonucleotides may be commercially obtained, or synthesized as desired. They may be assembled via enzymatic polymerization or ligation, for example. Random oligonucleotide fragments may be purified, for example by column separation, to isolate fragments of approximately the same or similar size (for example, about 300 nt-400 nt in size in the depicted example), and may be inserted into the cassettes. A pool of cassettes containing a vast variety of different unique identifier sequences (i.e. about 10⁷ in some examples) may be produced. The cassette may comprise primer annealing sequences (i.e. primer sites) and at least one sequencing primer annealing sequence (i.e. sequencing site), in a suitable arrangement so as to allow for amplification and/or sequencing of the DUID, such as the configuration as shown in FIG. 2. Primer and sequencing sites may be validated against the host genome to verify that there is no native amplification. Cassettes with different primers may be employed for different organisms or for different genomes, if desired. The cassette may comprise restriction enzyme array sites, and may be provided in the form of an insertion cassette carrier plasmid, for example. In certain embodiments, the cassette may be about 500 bp in length, and may be provided within a plasmid or carrier vector of about 1200 bp in size, for example.

Once the cassette is completed, an identified event may be triggered, and the cassette may be sent to a customer. The customer will typically be a producer, such as a grower in the agriculture industry. The producer may use suitable transformation and regeneration techniques to regenerate an organism of interest now comprising a cassette inserted into the genome. They may then generate a validation package containing at least a sample of genomic DNA from the transformed biological entity, which may be then sent back.

Validation:

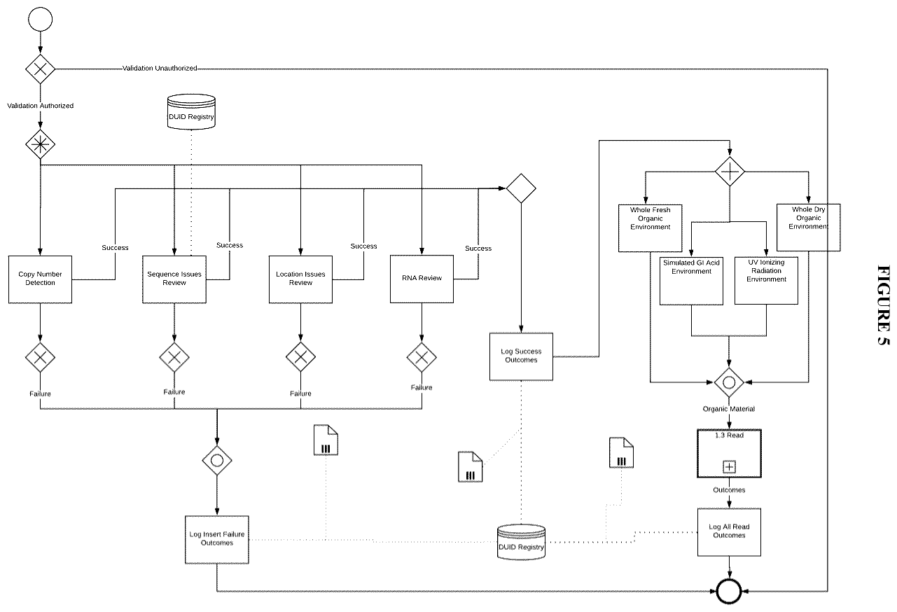

After receiving the validation package, authenticating the requester and checking for authorization, the validation process may begin. FIG. 5 outlines an example of a process for validation. The DUID may be validated for:

Stable Integration in the Host Nuclear Genome.
The DUID may be easily amplified from whole DNA extract.
The DUID sequence may be recoverable from the DUID cassette and within predictable specifications.

Unique Value Validity.
If the value is already present in the registry, the transformation event may be discarded.

Copy Number of the Integration.
Transformation events where there is more than one copy of the DUID may be discarded (although it is also contemplated that in some examples more than one DUID may be used).

Location of Integration
The DUID may be targeted to non-coding/intergenic regions to reduce the potential of the insertion affecting native coding regions.
The location of the DUID may also be mapped to a specific chromosome and chromosomal arm.

Non-Expression Assessment.
If there is any RNA expression of the DUID, the transformation event may be discarded.

The DUID may be amplified independently with both sets of primers (where more than one set is used, as in the example of FIG. 2, for example) and the random ID may be sequenced. This process may be repeated three times to mitigate sequencing errors in certain embodiments. The validation business service may utilize a succeed or fail stepwise flow for each of the cassette validation steps. This may reduce the cost of validation, in certain embodiments. If a failure occurs, the outcome may be logged. If each sequence validation succeeds, the results may be logged and the recall tests may begin.

In certain embodiments, such recall simulations may include introducing the organic material of interest to various environmental states. These environments may result in varying organic material, which may be subsequently passed to the read business service. In this example, there may be any of all of the following four parallel tests that may occur:

Whole Fresh Environment

Whole Dry Environment

Simulated GI Acid Environment
This may simulate the digestion of the material. may simulate the potential recall from fecal matter.

UV Ionizing Radiation Environment
This may simulate exposure of the organic material to sunlight or other food processing sterilization techniques such as gamma irradiation or e-beam sterilization.

Once these tests are complete, the generated organic material may independently be passed to, and trigger the read business service. Following the read business service, all outcomes may be logged. Not all organic material derived from these environmental state tests must be successfully read in order for validation to complete successfully, and such determinations may be made on a case-by-case basis, for example.

Post-Validation:

Depending on the outcome of validation, there may be a number of potential outflows. If the validation was unauthorized, the DUID service may be terminated, and relevant parties may be notified. If one of the sequence validation tests failed, a post-mortem review may be entered. The post-mortem review may attempt to identify the cause of the failure. Depending on that cause—there may be two outcomes (cassette error or transformation error)—the flow may either trigger a retry on the identification business service or request a transformation retry from the producer.

If the outcome of the validation business service is a validated event, the DUID registry (i.e. database) may be updated with relevant information. This event may also trigger a propagation approval message or notification, which may be received by the producer. They may then move forward with generating propagating material for the grower, who in turn may carry on with business as usual.

Pre-Read Supply-Chain Activity:

As described herein, the rest of the supply chain may continue with business as usual. Although, supply chain stakeholders may have the option of integrating the DUID into their existing processes. If they choose not to, the existence of the DUID may provide—at least—source-of-origin traceability. In certain embodiments, it is contemplated that the DUID may be integrated into existing barcodes. Note that in certain embodiments, the unique identifier (UID) portion of the DUID may be essentially a string of characters characterized by its nucleotides (A, T, G, C). In certain embodiments, if an explicit read is not required, they may independently track that DUID-ready organism using their own data capture technologies (for example, barcoding). This may result in an unconfirmed tracked event.

If a read event is required, the stakeholder in question may submit a request to the read business service. There may be two types of requests in this example. One may be mandatory and the other may be voluntary. The contents of the read package may depend on the type. For example, if the read request is mandatory, there may be specific requirements to be met in order to satisfy stakeholder requirements—e.g. organic material samples from particular dates.

Read:

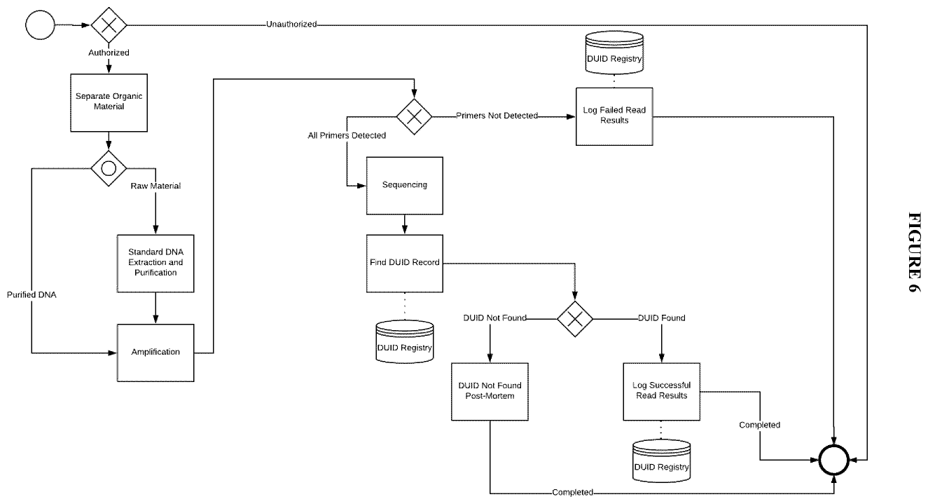

The read business service is shown in detail in FIG. 6. As with the other business services, authorization may be immediately checked for. Often, the read package may contain various types of organic material. Depending on that material, purification and/or amplification may be done. If the primers are detected, the sequencing (and in some cases UID decoding steps) may begin. If the primer is not detected, log the results and fail.

Once the UID has been sequenced and/or decoded, an attempt may be made to find all relevant data within the DUID registry. It is conceivable that in certain instances a DUID may not be found in the registry, in which case a post-mortem review may be conducted. This review may attempt to find the cause of error. On the other hand, if the DUID is found, the results may be logged and a read event may be created.

It is also possible that in certain embodiments an approved integration partner—e.g. the FDA—may make a request to the read business service. Some jurisdictions may have regulations, which may require the sharing of traceability data, for example.

Post-Read

After the read business services complete, a read data package may be generated and returned to the requesting stakeholder. The read package may contain all previous tracked events, validation results, and primer data. It may also contain contractual obligations that necessitated the use of the DUID in the first place. This may include KYC information for each party involved.

Supporting Systems:

There may be two supporting systems noted on the DUID global view diagram of this example. Neither of these may play an integral role to the overall process, but instead may function as interfaces and processors for the DUID Registry.

API: The API may function as an interface to the DUID registry. This may allow approved integration parties access to approved data. In some cases, they may be able to modify that data—see user access roles described above.

Stream Processor: The stream processor may read from the registry in real time and trigger functionality as a result. For example, if an unauthorized actor has requested the read business service, the DUID owner may be automatically notified, for example.

Accordingly, this Example describes in detail embodiments of a DUID system, methods, and compositions which may be used in accordance with the teachings provided herein. As will be understood, this Example is provided for illustrative purposes intended for the person of skill in the art, and is not intended to be limiting.

### EXAMPLE 2

### Stable DUID Integration in Yeast Species

Stable DUID Integration into Yeast Species:

In this example, stable integration of DUID into yeast species is described.

Methods and Materials for Stable DUID Integration into Yeast Species.

Overview:

This example describes approaches to design, integrate and validate DNA sequence-based unique identifiers (DUIDs) into model organism, yeast. These techniques involve the use of both laboratory yeast strains and industrial yeast strains. The methods herein validate utility and efficacy for DUID integration into a genome for the activities of traceability. These molecular biology laboratory methods include:

1. the in silico design of DUIDs, DUID vectors and DUID primers.

2. the method for stable genomic integration through yeast centromere plasmids (YCp).

3. the method for stable genomic integration through insertion into native yeast chromosomes.

4. the method DUID integration validation.

5. the method for DUID signal detection and signal detection limits.

It is contemplated that these methods are applicable to wide range of research and industrial yeast strains including prototrophic strains. The YCp approach allows for genome integration through cellular and nuclear management of the DUIDs constructs as independent chromosomes, through spindle association of the centromeric sequences built into the vector backbone. For insertion into native yeast chromosomes, four genomic sites were selected for minimal interference with the usual coding capacity and expression of genes within the genome. These sites included sub-telomeric regions that are generally regarded as heterochromatic where genes are typically silenced, and a euchromatic region with low coding capacity to act as a positive control. The insertion into native yeast chromosomes approach focuses on: 1) Co-transformation of a plasmid carrying antibiotic resistance for selection of transformants along with a linear fragment containing the DUID flanked by homologous regions flanking the selected target sites; and 2) CRISPR-based methods that target in integration site using specific guide RNAs (gRNAs) and specific homology repair templates (HRTs) that serve as templates for the Cas9-digested target PAM sites.

Construct and Vectors Design and Development:

DUID Construct Design

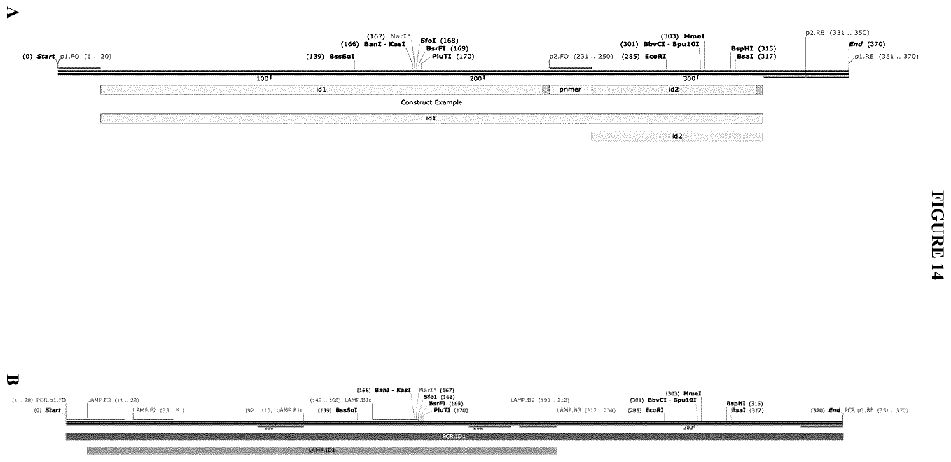

FIG. 14 shows maps of two 370 pb DUID constructs. A) DUID construct design for PCR and qPCR amplification. Construct is 370 pb. This DUID construct contains 2 forward primers and two reverse primers. There are two identifiers (ID1 and ID2). ID1 is ideal for PCR amplification. ID2 is ideal for qPCR amplification. B) A DUID construct design for loop-mediated isothermal amplification (LAMP) and PCR. This map includes primers for both PCR and LAMP. Aside from traditional amplification design decisions, note the features in pink, which are optional CAS PAM sites that allow for editing and detection of the DUID construct sequences using CRISPR-based systems. Such a PAM site may allow for editing of a DUID construct that has been integrated.

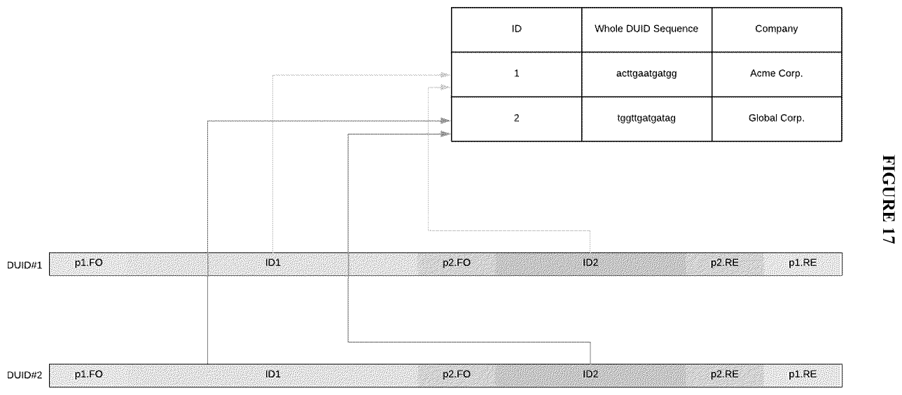

FIG. 17 depicts an ID to Registry mapping example as described herein. Note that this Figure depicts a simplified example, and it is contemplated that the whole DUID sequences would typically not be as short as those depicted in the table.

In the present example, there will not be more than one alignment of an ID sequence within the database. In this example the ID sequences are always unique to a single DUID construct, but a single DUID construct may have multiple ID sequences. However, an ID sequence may have one or more sections within it that is homologous to other DUID sequences. It is contemplated that there may be sequences within a DUID construct that may be used across DUID constructs; however, the IDs themselves should be unique, and by extension, the DUIDs will also be unique. This design decision to have homologous sections within ID sequences across any number of DUIDs may allow to version the DUIDs in a number of ways.

Homologous ID Section Example—One Homologous Section Across Three DUIDs:
There are a few reasons having homologous sequences across multiple identifiers may be desirable. In some cases, the identifier may have a homologous sequence for the purpose of providing a version associated with that identifier. The ability to version identifiers may allow users to reference an associated protocol that will inform how they may interact with the DUID. For example, a particular version of a DUID identifier may contain a public key within in the context of cryptography, which may inform subsequent interactions with the DUID in some meaningful way. In other cases, the homologous sequence may reference the system or entity that initially created the identifier, for example. The following Table depicts three DUIDs having such a homologous section—1:10 is homologous, 11:50 is unique in these exemplary DUID examples.

| | |
|---|---|
| HOMOLOGOUS | UNIQUE |
| NNNNNNNNNN | NNNNNNNNNNNNNNNNNNNNNNNNNNNNNNNNNNNNNNNN |

| | |
|---|---|
| DUID#1 | ACAACGGTCGTATGTATGCACTAGGTCAACAATAGGACATAGCCTTGTAG (SEQ ID NO: 2) |
| DUID#2 | ACAACGGTCGTTGTGTTCCGACAGGCTAGCATATTATCCTAAGGCGTTAC (SEQ ID NO: 3) |
| DUID#3 | ACAACGGTCGTACCGTCGGATTTGCTATAGCCCCTGAACGCTACATGTAC (SEQ ID NO: 4) |

YCp and Co-Transformation

The plasmid used for the co-transformation procedure was the yeast centromeric vector, YCp41K (Taxis & Knop, 2006). Four target sites for integration were identified: the sub-telomeric region of Chr6 and the euchromatic region of Chromosome 2 (Appendix C). The linear fragments targeting these sites contained the DUIDs flanked by 75 nt regions that are homologous to the regions flanking the respective integration sites (FIG. 14). The exact linear fragment sequences for each integration site is listed in Appendix D. These fragments were synthesized by Twist Bioscience (https://www.twistbioscience.com/) as both linear fragments and inserted into pRS41K vector (https://bip.weizmann.ac.il/plasmid/pics/106.jpg) and pRS42K (https://bip.weizmann.ac.il/plasmid/pics/109.jpg).

Generating Linear DUID Fragments for Co-Transformation

Linear DNA fragments for homologous recombination (HR) were created by PCR using the linear fragments generated by Twist Bioscience as templates. See Appendix A in “Co-transformation” below for specific fragments generated. The pRS41K-Chr6 and pRS41K-Euch on-boarded by Twist Bioscience, were used. The primers used to generate the HR fragment for the Chr6 target regions were Chr6_DUID F and DUID-synth R, and for the Euch target regions Euch DUID F and DUID-synth R, respectively (Appendix A). The PCR reaction composition (Table 2) and reactions conditions (Table 3) are detailed below.

TABLE 2
PCR reaction cocktail to create DUID template
using Phusion high fidelity polymerase

| | Volume | Volume | Final | |
|---|---|---|---|---|
| Component | (20 μL) | (50 μL) | Concentration | |
| 5X Phusion buffer | 4 μL | 10 μL | 1X | |
| 10 mM dNTP | 0.4 μL | 1 μL | 200 | μM each |
| Forward Primer | 1 μL | 2.5 μL | 0.5 | μM |
| Reverse Primer | 1 μL | 2.5 μL | 0.5 | μM |
| Template | 1 μL | 1 μL | 25 | ng |
| Phusion polymerase | 0.2 μL | 0.5 μL | 0.02 | U/μL |
| Water | 12.4 μL | 32.5 μL | | |

TABLE 3
Amplification parameters

| Step | Temp | Time | | Cycles |
|---|---|---|---|---|
| Initial Denaturation | 98 | 30 | s | 1 |
| Denaturation | 98 | 10 | s | 30 |
| Annealing | 60 | 20 | s | 30 |
| Extension | 72 | 15 | s | 30 |
| Final Extension | 72 | 10 | min | 1 |

| | | | |
|---|---|---|---|
| Hold | 4 | Hold | Hold |

Primers were validated and annealing temperatures were optimised in 20 μL reaction volumes. For generating HR linear integration fragments, 2× 50 μL reactions were performed and the products purified using a Qiagen PCR Purification kit (https://www.qiagen.com/ie/shop/pcr/qiaquick-pcr-purification-kit/). Purified DNA fragments were eluted in 50 μL of elution Buffer (10 mM Tris-Cl, pH 8.5). Products were verified by running 5 μL on a 1% agarose gel.

CRISPR Vector and HRT Generation:

CRISPR experiments were performed using the plasmid pCC-036 which contains CAS9 expressed by the TDH3p, the SNR52p to drive the expression of gRNAs, and hygR for selection on hygromycin as described in Krogerus et al., 2019. Three gRNAs were designed for each of the target integration sites using the Benchling software (https://www.benchling.com/). Primers containing the gRNA sequences (Appendix B) were used in PCR reactions using pCC-036 as template. The reaction compositions and conditions are outlined below (Table 3 & Table 4). These PCR reactions were transformed into E. coli. Plasmids were isolated from transformants and screened by sequencing to confirm correct clones (FIG. 14 and Appendix B). We constructed one gRNA clone for Chr6 (Chr6_2) and two for Euch (Euch_1; Euch_2). Primers were designed to be partially overlapping (˜8-10 bp non-overlapping each side) with the mutation in the middle of both primers and PCR was performed according to the protocol in Zheng, et al., 2004.

TABLE 4
PCR reaction cocktail to insert gRNAs and for PAM
site mutations - Phusion high fidelity polymerase

| | Volume | Volume | Final | |
|---|---|---|---|---|
| Component | (20 μL) | (50 μL) | Concentration | |
| 5X Phusion buffer | 4 μL | 10 μL | 1X | |
| 10 mM dNTP | 0.4 μL | 1 μL | 200 | μM each |
| Forward Primer | 1 μL | 2.5 μL | 0.5 | μM |
| Reverse Primer | 1 μL | 2.5 μL | 0.5 | μM |
| Template | 1 μL | 1 μL | 25 | ng |
| Phusion polymerase | 0.2 μL | 0.5 μL | 0.02 | U/μL |
| Water | 12.4 μL | 32.5 μL | | |

TABLE 5
Amplification conditions

| Step | Temp | Time | | Cycles |
|---|---|---|---|---|
| Initial Denaturation | 98 | 30 | s | 1 |
| Denaturation | 98 | 10 | s | 16 |
| Annealing | 55 | 20 | s | 16 |
| Extension | 68 | 24 | min | 16 |
| Final Extension | 68 | 1 | hour | 1 |

| | | | |
|---|---|---|---|
| Hold | 4 | Hold | Hold |

Primers were validated and optimised for annealing temperature using 20 μL reaction volumes; for integration, 5× 50 μL reactions were run followed by digestion of the vector with HindIII and BamHI (NEB). DNA was purified using Phenol/Chloroform/Isoamyl alcohol followed by ethanol precipitation in the presence of 0.1M ammonium acetate and glycogen. DNA was resuspended in 30 μL nuclease free water. Amplification was verified by running 5 μL on a 1% agarose gel.

TABLE 6
Reaction cocktail for insertion of gRNA sequences

| Component | Volume per reaction |
|---|---|
| HF buffer | 10 uL |
| Template DNA | 1 uL |
| F primer | 2.5 uL |
| R primer | 2.5 uL |
| dNTP | 1 uL |
| Phusion polymerase | 1 uL |
| Water | 32 uL |

TABLE 7
PCR amplification conditions

| Step | Temperature | Time | |
|---|---|---|---|
| HotStart | 94 | hold | |
| Initial Denaturation | 94 | 3 | min |
| *Denaturation | 94 | 1 | min |
| *Annealing | 52 | 1 | min |
| *Extension | 68 | 24 | min |
| Final Extension | 68 | 1 | hour |

| | | |
|---|---|---|
| End | 24 | hold |

*16 cycles of denature/anneal/extend

Following PCR, 10 uL was run on an 1% agarose gel (yield of SDM DNA is low with Phusion). A DpnI (NEB) digest of 10 μL of PCR amplicon in a 30 uL reaction volume was performed overnight at 37 C to linearize the methylated template DNA. Another 10 uL was separated on a gel and then 5 uL was transform into E. coli. Minipreps were performed on 12 colonies and sequences.

Yeast Transformations:

Transformation for YCp-DUID Vectors

A standard lithium acetate-based yeast transformation protocol as described in Mertenes et al. 2017 was used to transform both the CRISPR plasmid, as well as the repair template into the target strains and completed as described below.

1. Yeast was grown overnight in 100 mL YPD 2% growth medium at 30° C. to OD˜0.7-0.8.

2. Next, the yeast cell culture was centrifuged (3 minutes at 3000 rpm), washed once in sterile water and cells were resuspended into 200 μL 0.1 M lithium acetate solution.

3. After 10 minutes incubation at room temperature, 50 μL of the cell culture was mixed with 500 ng of plasmid, 300 μL PLI (142 M Polyethylene glycol, 0.12 M lithium acetate, 0.01 M Tris (pH7.5) and 0.001M EDTA) and 5 μL salmon sperm DNA (1 mg·mL-1). A negative control transformation containing no DNA (sterile water) was performed in parallel.

4. The yeast suspension was incubated for 30 minutes at 42° C.

5. Cells were centrifuged (3 minutes at 3000 rpm) and resuspended in fresh YPD2%, after which cells were recuperated for one overnight incubation at 30° C.

6. 200 uL of yeast suspension was plated onto YPD+G418 300 ug/mL, followed by a 2-day incubation at 30° C. 200 uL was also plated onto YPD containing no antibiotic to confirm cell viability following these treatments.

Co-Transformation

This method involved the generation of competent cells with lithium lcetate followed by DNA transformation using electroporation as described in Bernardi et al., 2019.

TABLE 8
Summary of transformations

| Strain | Vector | Linear DUID Construct |
|---|---|---|
| S288C (BY4743; 2n) | pRS41K | S288C_Euch_Chr2 |
| | | S288C_Chr6 |
| Vermont (Ale yeast; 4n) | pRS41K | Verm_Chr2 |
| | | Verm _Chr6 |
| French Saison (Ale yeast; 4n) | pRS41K | FrenSais_Chr2 |
| | | FrenSais _Chr6 |

Steps in Co-Transformation:

1. Cells were grown in 100 mL of YPD with shaking to the desired growth phase (based on growth curves or OD).

2. Cells were harvested at mid-log growth (OD₆₀₀=0.7-0.8). Culture was spun down culture and the supernatant discarded.

3. Pellet was washed once with sterile water. Culture was spun down culture and the supernatant discarded, and resuspend in 25 mL of 0.1M lithium acetate/10 mM DTT/10 mM TE solution (Tris HCl:EDTA=10:1). Culture was incubated for 1 h at room temperature. Culture was spun down culture and the supernatant discarded.

4. Note: If working with flocculant strains make sure to invert the tubes a few times every 10 mins to prevent cells from settling to the bottom of the tube.

5. Pellet was washed with 25 mL of ice-cold distilled sterile water and the culture spun down at 4 C and the supernatant removed. Step was repeated (for a total of two washes).

6. Pellet was washed with 10 mL of ice-cold sorbitol, spun down at 4 C, and removed the supernatant. Resuspend the pellet in 100 uL of ice-cold sorbitol.

7. Used 100 uL of the cell suspension for transformation.

8. Mixed 15 uL (1 μg of pRS41K [YCp plasmid]+1 μg linear DUID fragment; 1:10 molar ratio; and 1:20 molar ratio) of the transforming DNA with the cell suspension and incubated on ice for 5 mins.

9. Cell suspensions were electroporated with 1.8 kV in 0.1 cm cuvettes.

10. 1 mL of cold sorbitol was added to the electroporation cuvette and mixed with the cell suspension. Suspension was transferred to a tube with 300 uL of YPD.

11. Note: If using an antibiotic marker incubate the suspension for 3 h at 30 C to allow for antibiotic expression to occur. * do not add the antibiotic to this culture. It will kill all of your cells since they are not yet expressing the plasmid that provides them with antibiotic resistance.*

12. 100 uL of the transformed culture was plated onto selective (YPD+300 mg/L G418) plates and incubated at 30 C for 5 days for colonies to appear.

13. Transformed cultures were plated onto YPD plates without any markers/antibiotics as well, to ensure that the cells were alive.

Transformation with CRISPR Vectors and HRT:

A standard lithium acetate-based yeast transformation protocol was used to transform both the CRISPR plasmid, as well as the repair template into the target strains as described in Mertens, et al., 2019. This protocol described below is based on standard transformation procedures where the cells are made competent by treatment with LiOAc solution after which cells are incubated with DNA molecules (plasmid and repair template) and carrier DNA (salmon sperm DNA) prior to a heat shock to take up the DNA. Following recuperation, the cells are plated on hygromycin to select against all non-transformed cells. Plating on YPD without hygromycin showed the growth of cells following the transformation procedure; e.g. the procedure itself did not kill the cells. Transforming the CRISPR plasmid without the HRT, should kill the cells as the DSB will not repair; this will confirm the successful function of the CRISPR plasmid meaning Cas9 is expressed and the gRNAs target Cas9 to the genome. Transforming the CRISPR plasmid along with the HRT should repair the DSB and support cell growth.

The plasmid pCC-036_Chr6_2/Chr6_HRT and pCC-036_Euch_1/Euch-HRT were the respective combinations of DNA molecules transformed into yeast strains S288c, Vermont and French Saison. The following protocol was used.

1. yeast was grown overnight in 5 mL YPD at 30° C., 200 rpm, after which 1 mL of the pre-culture was transferred to 50 mL YPD and incubated for an extra 4 hours (30° C., 200 rpm).

2. Next, the yeast cell culture was centrifuged (3 minutes at 3000 rpm) and cells were resuspended into 200 μL 0.1 M lithium acetate solution.

3. After 10 minutes incubation at room temperature, 50 μL of the cell culture was mixed with 500 ng plasmid, in which the corresponding sgRNA was cloned with and without 5 to 25 μg (adjusted protocol) HRT DNA, 300 μL PLI (142 M Polyethylene glycol, 0.12 M lithium acetate, 0.01 M Tris (pH7.5) and 0.001M EDTA) and 5 μL salmon sperm DNA (1 mg·mL⁻¹).

4. Incubated for 30 minutes at 42° C.

5. Cells were centrifuged (3 minutes at 3000 rpm) and resuspended in fresh YPD, after which cells were recuperated for one overnight at 30° C.

6. 200 μL volumes of yeast suspension were plated onto YPD containing hygromycin at 300 mg/L, followed by a 3-5 day incubation at 30° C.

Screening Transformants:

Genomic DNA Extraction Protocol

1. Replica plate the co-transformation plates onto YPD+G418 (300 mg/L)

2. Section the master plate into 4-8 colonies per section. Scrape the colonies into a sterile tube with 3 mL YPD and grow overnight with shaking at 30° C.

3. Pellet 2 mL culture in a 2 mL screw cap tube

4. Wash once with 1 mL MQ water

5. Resuspend in 200 μL Breaking buffer (2% TX-100, 1% SDS, 100 mM NaCl, 100 mM Tris pH 7.5)

6. Add 200 uL glass beads and 200 uL of Phenol/Chloroform/Isoamyl alcohol

7. Vortex on high for 3 min

8. Centrifuge at max for 5 min

9. Transfer top aqueous layer to a clean microcentrifuge tube

10. Add 1 mL 100% EtOH and mix by inversion

11. Centrifuge at max for 3 min

12. Decant ethanol, dry pellet and resuspend in 400 uL 1× TE

13. Add 30 uL 1 mg/mL RNase A

14. Incubate for 5 min at 37

15. Add 10 uL 4M Ammonium acetate and 1 mL 100% EtOH. Mix by inversion

16. Centrifuge 3 min at max. Wash pellet in 1 mL 70% EtOH and allow to dry

17. Resuspend in 100 uL of water Identification of Integrants & PCR Screening of Transformants:

The gDNA isolated as described above served as a template for PCR reactions using primers that bind the genomic DNA in specific regions up and downstream of the homologous regions of the HRT that flank the target integration site (see Appendix A for primer details; the reaction composition and conditions are outlined in tables 9 & 10 below). For the euchromatic target integration site on Chr2, primers Euch_Seq F/R were used, and for the Chr6 subtelomeric heterochromatic target integration site, primers Chr6_Seq F/R were used. These primers yielded a ˜600 bp DNA fragment from gDNA without any insertion at the integration site. With integration, this fragment size will increase to ˜970 bp. The controls included reactions without any gDNA template, and gDNA isolated from untransformed strains (like S288c/BY4743). PCR reactions were resolved with gel electrophoresis using GeneRuler 100 bp Plus molecular weight marker to confirm the size of the generate DNA fragments.

Once integrants were confirmed, the correct integration was validated with PCR primers of which bind the genome outside the integrating fragment and another that binds within the transformed fragment. These primers yield a DNA fragment if the integration occurred at the correct target site and no DNA fragment if integration did not occur.

DNA fragments generated by both the integration confirmation and validation assays are sequences to confirm integration.

TABLE 9
DUID screening PCR reaction cocktail

| Component | Quantity/20 uL rxn | |
|---|---|---|
| Standard buffer | 4 | uL |
| Template DNA | ^{~}100-400 ng (1 uL) | |
| F primer | 1 | uL |
| R primer | 1 | uL |
| dNTP | 0.4 | uL |
| OneTaq HotStart polymerase | 0.1 | uL |
| Water | 12.6 | uL |

TABLE 10
DUID screening PCR reaction program

| Step | Temperature | Time | |
|---|---|---|---|
| Initial Denaturation | 98 | 3 | min |
| *Denaturation | 98 | 30 | s |
| *Annealing | 60 | 30 | s |
| *Extension | 68 | 1 | min |
| Final Extension | 68 | 10 | min |

| | | |
|---|---|---|
| End | 12 | hold |

*Do 30 cycles of denature/anneal/extend

Following PCR, 10 uL of the reaction was resolved on a 1% agarose gel (1× TAE, containing SYBR Safe nucleic acid stain).

Confirmation of Insertion Copy Number and Location:

We performed WGS on the parent and integrants to confirm insertion copy number and to identify the presence of any off-target integration events. We will combine short-read (Illumina) and long-read (PacBio) sequencing data to assemble the full genomes of both the parent and transformed strains. The combination of these two approaches will provide an overall genome structure of the integrant and hence identify if multiple insertions occurred or if there were any off-target integration events. The whole genomes of integrant(s) and parent strain(s) will be sequenced by Genome Québec (Montreal, Canada) as previously described (Preiss et al., 2018). In brief, DNA will be isolated and used as templates for library constructions for Illumina and PacBio applications. Sequencing reads will be quality-analysed with FastQC (version 0.11.5) (Andrews, 2010) and trimmed and filtered with Trimmomatic (version 0.36) (Bolger, Lohse, & Usadel, 2014). Reads will be aligned to a S. cerevisiae S288c (R64-2-1) reference genome using SpeedSeq (0.1.0) (Chiang et al., 2015). Quality of alignments will be assessed with QualiMap (2.2.1) (Garcia-Alcalde et al., 2012). Variant analysis will be performed on aligned reads using FreeBayes (1.1.0-46-g8d2b3a01) (Garrison & Marth, 2012). Variants in all strains will be called simultaneously (multi-sample). Prior to variant analysis, alignments will be filtered to a minimum MAPQ of 50 with SAMtools (1.2) (Li et al., 2009). Annotation and effect prediction of the variants will be performed with SnpEff (1.2) (Cingolani et al., 2012). Copy number variations of chromosomes and genes will be estimated based on coverage with Control-FREEC (11.0) (Boeva et al., 2012). Statistically significant copy number variations will be identified using the Wilcoxon Rank Sum test (p<0.05). The median coverage and heterozygous SNP count over 10,000 bp windows will be calculated with BEDTools (2.26.0) (Quinlan & Hall, 2010) and visualized in R.

Determining Expression of DUID in Integrants Using Droplet Digital PCR:

We will use droplet digital PCR (ddPCR), which allows for the quantification of the absolute number of molecules within the sample. This specifically allows for the quantification of copy numbers or low expressing genes. The procedure involves the isolation of gDNA-free RNA from yeasts, followed by cDNA synthesis and ultimately the generation of

S288c with and without the pRS41K-Euch plasmid and the integrants strain will be grown in YPD in triplicate. RNA will be extracted with the commonly used hot acid phenol method (COLLART AND OLIVIERO 2001) and quantified with a NanoDrop 2000C spectrophotometer (NanoDrop Technologies Inc.). RNA samples will be treated with RapidOut DNA Removal Kit (Thermo Fisher), tested for DNA contamination and assessed for quality using an Agilent 2100 Bioanalyzer. RNA (1000 ng per sample) will be used to create cDNA using High Capacity cDNA Reverse Transcription kit (Applied BioSystems).

These samples along with diluted pRS41K-Euch will be submitted for ddPCR analyses at the Genomics Facility at the University of Guelph. These samples, along with a “No Template Control”, will be used as templates in ddPCR reactions using ddPCR EvaGreen Supermix (enables emulsification) on all reactions along with qPCR primers for the DUID, GAT3 (low expresser control) and ACT1 (high expressor control). Nanoliter-sized droplets were generated on the AutoDG™ Instrument (Bio-Rad), then PCR amplification will be performed using a C1000 Touch Thermal Cycler (Bio-Rad). After PCR cycling, the ddPCR plate will be read in the QX200 Droplet Reader from Bio-Rad and the data will be analyzed using the QuantaSoft Analysis Pro software version 1.0.596 (Bio-Rad Laboratories).

Protocol for LOD/LOQ Analysis:

gDNA prepared using gDNA isolation protocol used for screening for insertion. Vectors were prepared from DH5a K12 cultures grown in the presence of Ampicillin, using QiaQuick Miniprep kit.

Standard PCR Protocol

Primers: S288C DUID F and R

Dilution series: 100 ng, 10 ng, 1 ng, 100 pg, 10 pg, 1 pg, 100 fg, 10 fg, 1 fg, 100 ag

TABLE 11
PCR reactions performed using GoTaq
polymerase in 20 uL reactions

| Component | Quantity/20 uL rxn |
|---|---|
| 2X Green MM | 10 uL |
| Template DNA | Per dilution series |
| F primer | 1 uL |
| R primer | 1 uL |
| Water | 7 uL |

TABLE 12
PCR reactions conditions

| Step | Temperature | Time | |
|---|---|---|---|
| Initial Denaturation | 98 | 3 | min |
| *Denaturation | 98 | 30 | s |
| *Annealing | 60 | 30 | s |
| *Extension | 72 | 30 | s |
| Final Extension | 72 | 10 | min |

| | | |
|---|---|---|
| End | 12 | hold |

*30 cycles of denature/anneal/extend

Following PCR, 10 uL of the reaction was separated on a 1% agarose gel (1× TAE, containing SYBR Safe nucleic acid stain).

Quantitative PCR (qPCR) Protocol:

qPCR reactions performed by university of Guelph AAC Genomics facility using SensiFAST Hi-ROX SYBR Master Mix in StepOnePlus Real-Time PCR system. qPCR cycling conditions are described in Table 12. Analysis was completed using Applied Biosystems StepOnePlus software. gDNA was prepared using gDNA isolation protocol described above. Control DUID vector was prepared from DH5α K12 cultures grown in the presence of Ampicillin, using QiaQuick Miniprep kit.

Amplification was performed on both plasmid and YCp yeast gDNA samples using the following primer and across the dilutions series.

Primers: S288C DUID qPCR F and R

Dilution series: 100 ng, 10 ng, 1 ng, 100 pg, 10 pg, 1 pg, 100 fg, 10 fg, 1 fg, 100 ag

Results & Discussion

Transformation Validation:

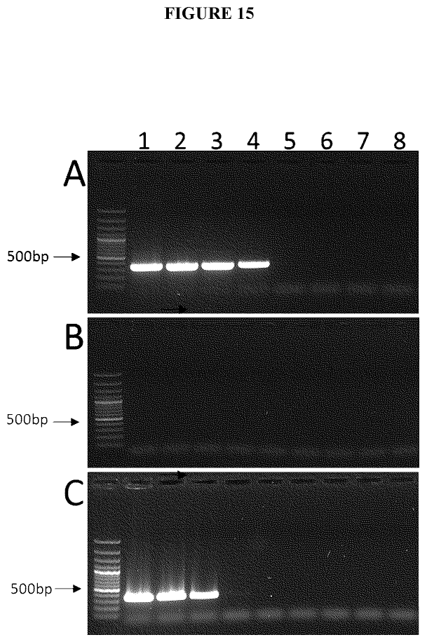

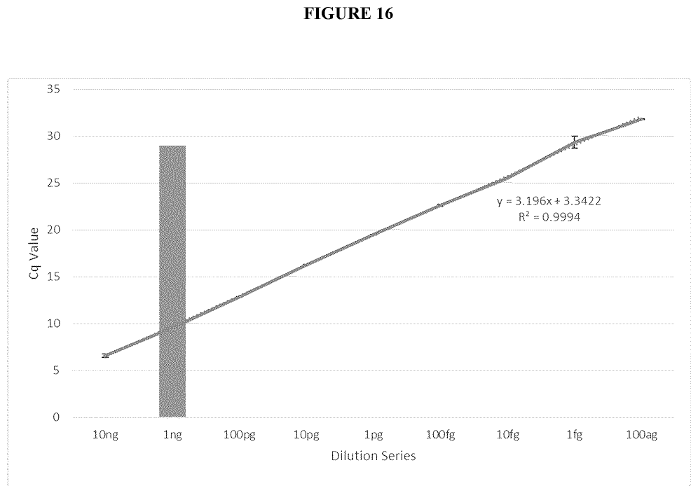

A DUID was stably transformed into the yeast strain (BY4743) genome via the YCp vector. Transformed yeast were cultured and genomic DNA was extracted as described above. Stable integration Cingolani P, Platts A, Wang le L, Coon M, Nguyen T, Wang L, Land S J, Lu X, Ruden D M. A program for annotating and predicting the effects of single nucleotide polymorphisms, SnpEff: SNPs in the genome of Drosophila melanogaster fly strain (w1118; iso-2; iso-3, Austin. 2012 April-June; 6(2):80-92. doi: 10.4161/fly.19695. PMID: 22728672; PMCID: PMC3679285.on) was verified by end-point PCR (FIG. 15, B1-B3) and qPCR (FIG. 16). End-point analysis of PCR amplification of using DUID recall primers that flank the DUID construct yielded positive amplification for both YCp-DUID vector (FIG. 15A) and yeast genomic DNA extractions from cells transformed with the YCp-DUID vectors (FIG. 15C). The 370 bp band indicating DUID amplification was clearly visible when quantities of 100 pg-100 ng of YCp-DUID vector were used as template (FIG. 15A lanes 1-4), while there was no detectable amplification from any input DNA quantity with untransformed BY4743 genomic DNA (FIG. 15B lanes 1-8). Similar assays using total DNA isolated from cells transformed with YCp-DUID vector resulted in positive amplification in the range of 1-100 ng of input DNA, with a very faint signal from 100 pg of input DNA (FIG. 15C lanes 1-4), indicating that DUID present at 1-2 copies per cell, a copy number reflective of that of chromosomal features, can be easily detected within yeast gDNA isolates using standard end-point PCR procedures.

FIG. 15 shows detection of YCp-DUID in yeast genomic DNA by end-point PCR. PCR amplification was performed using (A) YCp-DUID vector and (B) gDNA extracted from BY4743 and (C) yeast strain BY4743 transformed with YCp-DUID vector as templates with DUID recall primers. Reactions were performed using serially diluted DNA template with input quantities of (1) 100 ng, (2) 10 ng, (3) 1 ng, (4) 100 pg, (5) 10 pg, (6) 1 pg, (7) 100 fg and (8) 10 fg and resolved on an 1% agarose gel with GeneRuler™ 100 bp Plus Ready-to-use Ladder as standard.

LOD/LOQ Analysis:

Quantitative real-time PCR was performed using serial 10-fold dilutions of purified YCp-DUID vector (FIG. 16); in these assays DUID amplification was detected at all measured concentrations, indicating that DUIDs can be reliably identified at concentrations as low as 500 ag. A standard curve was generated by plotting the mean Cq values vs known DNA input concentrations using MS Excel. Based on this standard curve, the R² was calculated as 0.9993 with 105.5% primer efficiency (calculated using Agilent QPCR Standard Curve to Slope Efficiency calculator, https://www. chem.agilent.com/store/biocalculators/calcSlopeEfficiency.jsp?_requestid=1116919), indicating that the reaction efficiency was within the accepted standards for high quality qPCR analyses (https://www.gene-quantification.de/roche-rel-quant.pdf). A similar qPCR assay conducted using DNA isolated from BY4743 transformed with YCp-DUID indicated that DUID could be detected within 50 ng of total yeast DNA, with a mean Cq value of 29.02. This Cq value was plotted against the standard curve described above (FIG. 3; orange bar). These results validate that the DUID recall methods can amplify DUID from a yeast cell culture matrix.

These results have demonstrated:
1. DUIDs can be successfully designed and stably transformed into yeast
2. For the purpose of traceability, DUIDs can be recalled from a biological matrix through both standard end-point PCR and qPCR techniques.

FIG. 16 shows detection of DUID within yeast total DNA extracts. Quantitative real-time PCR was performed on serial 10-fold dilutions of YCp vector, ranging from 50 ng-500 ag and used to generate a standard curve (blue line) using MS Excel. Results of a similar qPCR experiment using DNA derived from BY4743 transformed with YCp-DUID vector were plotted (orange bar) and compared with standard curve values to quantify detection of DUID within yeast biomass.

APPENDIX A
Primers used to generate linear transformation fragments or recall DUIDs

| Primer | Sequence (5′-3′) | Use |
|---|---|---|
| Chr6_DUID F | CATTCCGCCTGACCTGGAG | Synthesis of linear fragments for co- |
| | (SEQ ID NO: 5) | transformation from Twist fragments |
| Euch_DUID F | CATTCCGCCTGACCCCTTAAT | Synthesis of linear fragments for co- |
| | (SEQ ID NO: 6) | transformation from Twist fragments |
| DUID_synth R | CACTGAGCCTCCACCTAGC | Synthesis of linear fragments for co- |
| | (SEQ ID NO: 7) | transformation from Twist fragments |
| Chr6_Seq F | AAGCGTAATTCCGAAAGGCA | Chr6 flanking primer: Binds genome 5′ of |
| | (SEQ ID NO: 8) | integration site |
| Chr6_Seq R | TGCATACGCTTCTCTCGACT | Chr6 flanking primer: Binds genome 3′ of |
| | (SEQ ID NO: 9) | integration site |
| Euch_Seq F | CAGAAATGGACAAGGAGATATGTGA | Euch flanking primer: Binds genome 5′ of |
| | (SEQ ID NO: 10) | integration site |
| Euch_Seq R | TTGAGTACCTGGCCAATGGAG | Euch flanking primer: Binds genome 3′ of |
| | (SEQ ID NO: 11) | integration site |
| S288C_recall F | GCTGATGGTTTAGGCGTACA | Recall of S288c DUID |
| S288C_recall R | (SEQ ID NO: 12) | |
| | CCCTGGAAATGCACTTGGTC | |
| | (SEQ ID NO: 13) | |
| S288C_qPCR F | TGGTCGTTTGGCTGTAGAGA | qPCR of S288c DUID |
| S288C_qPCR R | (SEQ ID NO: 14) | |
| | CGTATAGAGCGGGTCATCGA | |
| | (SEQ ID NO: 15) | |
| Verm_recall F | ACTCTCCCATTAGTCGGCAG | Recall of Vermont DUID |
| Verm_recall R | (SEQ ID NO: 16) | |
| | AAGACCGCTTTGTTCCGACA | |
| | (SEQ ID NO: 17) | |
| Verm_qPCRF | GGCCCTATCAGTACAGCAGT | qPCR of Vermont DUID |
| Verm_qPCRR | (SEQ ID NO: 18) | |
| | AGTGCTGGCGAGAGAATGAA | |
| | (SEQ ID NO: 19) | |
| FrenSais_recall F | GCGTACAATGCCCTGAAGAA | Recall of French Saison DUID |
| FrenSais_recall R | (SEQ ID NO: 20) | |
| | CTCCCTGGAAATGCACTTGG | |
| | (SEQ ID NO: 21) | |
| FrenSais_qPCR F | AGCGGGTCATCGAAAGGTTA | qPCR of French Saison DUID |
| FrenSais_qPCR R | (SEQ ID NO: 22) | |
| | CACTTGGTCGTTTGGCTGTA | |
| | (SEQ ID NO: 23) | |

APPENDIX B
CRISPR Primers used to clone gRNAs or the mutate PAM sites in the HRTs

| Primer | Sequence (5′-3′) | Use |
|---|---|---|
| Chr6_1 F | AGTTGCAAAAAACAAGGGAAGTTTTAGAGCTAGAAATAGCAAGTTAAA | gRNA insertion into pCC- |
| | ATAAGG | 036:Chr6_l |
| | (SEQ ID NO: 24) | |
| Chr6_1 R | TCCCTTGTTTTTTGCAACTGATCATTTATCTTTCACTGCGGAG | |
| | (SEQ ID NO: 25) | |
| Chr6_2 F | GAGATCTTGTTTTATCATGAGTTTTAGAGCTAGAAATAGCAAGTTAAAA | gRNA insertion into pCC- |
| | TAAGG | 036:Chr6_2 |
| | (SEQ ID NO: 26) | |
| Chr6_2 R | CATGATAAAACAAGATCTCGATCATTTATCTTTCACTGCGGAG | |
| | (SEQ ID NO: 27) | |
| Chr6_3 F | AGATCTTGTTTTATCATGAGGTTTTAGAGCTAGAAATAGCAAGTTAAAA | gRNA insertion into pCC- |
| | TAAGG | 036:Chr6_3 |
| | (SEQ ID NO: 28) | |
| Chr6_3 R | TCATGATAAAACAAGATCTGATCATTTATCTTTCACTGCGGAG | |
| | (SEQ ID NO: 29) | |
| Euch_1 F | ATACTAAGTCAACATCAAGGGTTTTAGAGCTAGAAATAGCAAGTTAAA | gRNA insertion into pCC- |
| | ATAAGG | 036: Euch_l |
| | (SEQ ID NO: 30) | |
| Euch_1 R | CCTTGATGTTGACTTAGTATGATCATTTATCTTTCACTGCGGAG | |
| | (SEQ ID NO: 31) | |
| Euch_2 F | GAAATACTAAGTCAACATCAGTTTTAGAGCTAGAAATAGCAAGTTAAAA | gRNA insertion into pCC- |
| | TAAGG | 036: Euch_2 |
| | (SEQ ID NO: 32) | |
| Euch_2 R | TGATGTTGACTTAGTATTTCGATCATTTATCTTTCACTGCGGAG | |
| | (SEQ ID NO: 33) | |
| Euch_3 F | TCTTGGCTTTTACAACCGAGGTTTTAGAGCTAGAAATAGCAAGTTAAAA | gRNA insertion into pCC- |
| | TAAGG | 036: Euch_3 |
| | (SEQ ID NO: 34) | |
| Euch_3 R | CTCGGTTGTAAAAGCCAAGAGATCATTTATCTTTCACTGCGGAG | |
| | (SEQ ID NO: 35) | |
| Chr6_1 mut F | CAAGGGAAACGAAAATCAATCAAATTAG | Mutate the PAM site in |
| | (SEQ ID NO: 36) | HRT for Chr6_1 |
| Chr6_1 mut R | GATTGATTTTCGTTTCCCTTGTTT | |
| | (SEQ ID NO: 37) | |
| Chr6_2_3 mut R | CCTCCACCTAGCCTCCGCTCATGATAAA | Mutate the PAM sites in |
| | (SEQ ID NO: 38) | HRT for Chr6_2 and |
| Chr6_Vector R | GCTGCAGCCCCTCATGATAA | Chr6_3 |
| | (SEQ ID NO: 39) | |
| Euch_1 mut F | GAGGAAATACTAAGTCAACATCAAGGTCGCA | Mutate the PAM site in |
| | (SEQ ID NO: 40) | HRT for Euch_1 |
| Euch_1 mut R | TGCGACCTTGATGTTGACTTAGTATTTCCTCTCGG | |
| | (SEQ ID NO: 41) | |
| Euch_2 mut F | CTAAGTCAACATCAACGTGGCA | Mutate the PAM site in |
| | (SEQ ID NO: 42) | HRT for Euch_2 |
| Euch_2 mut R | TGCCACGTTGATGTTGACTTAGTATTTCC | |
| | (SEQ ID NO: 43) | |
| Euch_3 mut F | TACAACCGAGACGAAATACTAAGTCAACATC | Mutate the PAM site in |
| | (SEQ ID NO: 44) | HRT for Euch_3 |
| Euch_3 mut R | CTTAGTATTTCGTCTCGGTTGTAAAAGCCA | |
| | (SEQ ID NO: 45) | |
| Euch_Vector R | GGGCTGCAGTCAGCAGAT | |
| | (SEQ ID NO: 46) | |

APPENDIX C
Homologous Recombination Constructs
Whole sequences including homology arms for use in Homologous
Recombination Yeast chromosomal integration Sites
ChrII:809650..809799
Left Homology:
ACACAAACTGGCGTAGAAGGGGAAACGGAAATAGGGTCTGACGAGGAAGATAGCA
TAGAGGACGAGGGAAGCAGC (SEQ ID NO: 47)
Right Homology:
AGTGGAGGAAATAGTACGACAGAAAGACTAGTACCACACCAGCTGAGGGAACAAG
CAGCCAGACATATAGGAAAA (SEQ ID NO: 48)
ChrVI:261123..261272
Left Homology:
TGGAGTTGCAAAAAACAAGGGAAAGGAAAATCAATCAAATTAGAATTAAGGTTTTT
TTTGGACAGTGCAGCGTCA (SEQ ID NO: 49)
Right Homology:
ATGCGCACGTAATGGCTTCGAAGAAAAAAAGAAGGCAAATACAATGAAGCTGAGAT
CTTGTTTTATCATGAGGGG (SEQ ID NO: 50)
ChrXIV:764201..764350
Left Homology:
CAAATAAATTAGGCTCATAACCGTAATTTTATTCGAGACATTTTTGGTTACTTCAAA
ATATTGTTATTATATAAA (SEQ ID NO: 51)
Right Homology:
GATCATATAAAGTTCTTGGACAAGATTGGATACATTTAGTTTTATTTTTGAAAATCAC
AAAGATGAAACAAAATA (SEQ ID NO: 52)

APPENDIX D
Construct Components and Lengths

| Total Size | 520 | b |
|---|---|---|
| Left Homology Arm | 75 | b |
| Forward Primer 1 | 20 | b |
| ID1 | 210 bp (including pam flanking on 3′) | |
| Forward Primer 2 | 20 | b |
| ID1 | 80 bp (including pam flanking on 3′) | |
| Reverse Primer 2 | 20 | b |
| Reverse Primer 1 | 20 | b |
| Right Homology Arm | 75 | b |

S288c

ID1 (PCR Primers)

| OLIGO | start | len | tm | gc% | any_th | 3′_th | hairpin | seq |
|---|---|---|---|---|---|---|---|---|
| LEFT-PRIMER | 66 | 20 | 57.98 | 50 | 0.00 | 0.00 | 0.00 | GCTGATGGTTTAGGCGTACA |
| | | | | | | | | (SEQ ID NO: 53) |
| RIGHT-PRIMER | 437 | 20 | 59.11 | 55 | 0.00 | 0.00 | 0.00 | CCCTGGAAATGCACTTGGTC |
| | | | | | | | | (SEQ ID NO: 54) |

ID2 (qPCR Primers)

| OLIGO | start | len | tm | gc% | any_th | 3′_th | hairpin | seq |
|---|---|---|---|---|---|---|---|---|
| LEFT-PRIMER | 301 | 20 | 58.85 | 55 | 10.15 | 0.00 | 0.00 | CGTATAGAGCGGGTCATCGA |
| | | | | | | | | (SEQ ID NO: 55) |
| RIGHT-PRIMER | 422 | 20 | 58.67 | 50 | 0.00 | 0.00 | 0.00 | TGGTCGTTTGGCTGTAGAGA |

(SEQ ID NO: 56)

s288c-Chromosome 2
ACACAAACTGGCGTAGAAGGGGAAACGGAAATAGGGTCTGACGAGGAAGATAGCA
TAGAGGACGAGGGAAGCAGCGCTGATGGTTTAGGCGTACACGAGATCCTGGTTCAA
CGCGCTGCAAACCTACCCTGCTCCAAACTGCTGTTCAACGCCACTCTAACTGGCAGG
CAAATTATTAGTTTCTAAGTTCCCCAGGTGCTGAAGAGCAGTCATTCAACGCCCTCA
GATCATCCCGGCAAGTTGGCTGGCGCGTTTGTCCGGAGGATCGTGTCGTACAACAAC
CATCTGACTATCAACCCTCCaggCGTATAGAGCGGGTCATCGATGCGCTCAGGGAAC
AACAACGATAGGCCTGCGGCTGGTCACCATCGGGAAGTTTTGCTGGAGATCTGCTGC
TGTAGGaggTCTCTACAGCCAAACGACCAGACCAAGTGCATTTCCAGGGAGTGGAGG
AAATAGTACGACAGAAAGACTAGTACCACACCAGCTGAGGGAACAAGCAGCCAGA
CATATAGGAAAA (SEQ ID NO: 57)
s288c-Chromosome 6
TGGAGTTGCAAAAAACAAGGGAAAGGAAAATCAATCAAATTAGAATTAAGGTTTTT
TTTGGACAGTGCAGCGTCAGCTGATGGTTTAGGCGTACACGAGATCCTGGTTCAACG
CGCTGCAAACCTACCCTGCTCCAAACTGCTGTTCAACGCCACTCTAACTGGCAGGCA
AATTATTAGTTTCTAAGTTCCCCAGGTGCTGAAGAGCAGTCATTCAACGCCCTCAGA
TCATCCCGGCAAGTTGGCTGGCGCGTTTGTCCGGAGGATCGTGTCGTACAACAACCA
TCTGACTATCAACCCTCCaggCGTATAGAGCGGGTCATCGATGCGCTCAGGGAACAA
CAACGATAGGCCTGCGGCTGGTCACCATCGGGAAGTTTTGCTGGAGATCTGCTGCTG
TAGGaggTCTCTACAGCCAAACGACCAGACCAAGTGCATTTCCAGGGATGCGCACGT
AATGGCTTCGAAGAAAAAAAGAAGGCAAATACAATGAAGCTGAGATCTTGTTTTAT
CATGAGGGG (SEQ ID NO: 58)
s288c-Chromosome 14
CAAATAAATTAGGCTCATAACCGTAATTTTATTCGAGACATTTTTGGTTACTTCAAA
ATATTGTTATTATATAAAGCTGATGGTTTAGGCGTACACGAGATCCTGGTTCAACGC
GCTGCAAACCTACCCTGCTCCAAACTGCTGTTCAACGCCACTCTAACTGGCAGGCAA
ATTATTAGTTTCTAAGTTCCCCAGGTGCTGAAGAGCAGTCATTCAACGCCCTCAGAT
CATCCCGGCAAGTTGGCTGGCGCGTTTGTCCGGAGGATCGTGTCGTACAACAACCAT
CTGACTATCAACCCTCCaggCGTATAGAGCGGGTCATCGATGCGCTCAGGGAACAAC
AACGATAGGCCTGCGGCTGGTCACCATCGGGAAGTTTTGCTGGAGATCTGCTGCTGT
AGGaggTCTCTACAGCCAAACGACCAGACCAAGTGCATTTCCAGGGGATCATATAAA
GTTCTTGGACAAGATTGGATACATTTAGTTTTATTTTTGAAAATCACAAAGATGAAA
CAAAATA (SEQ ID NO: 59)
Vermont
ID1 (PCR)

| OLIGO | start | len | tm | gc% | any_th | 3′_th | hairpin | seq |
|---|---|---|---|---|---|---|---|---|
| LEFT-PRIMER | 173 | 20 | 58.88 | 55.00 | 0.00 | 0.00 | 0.00 | ACTCTCCCATTAGTCGGCAG |
| | | | | | | | | (SEQ ID NO: 60) |
| RIGHT-PRIMER | 541 | 20 | 60.18 | 50.00 | 0.00 | 0.00 | 0.00 | AAGACCGCTTTGTTCCGACA |
| | | | | | | | | (SEQ ID NO: 61) |

ID2 (qPCR Primers)

| OLIGO | start | len | tm | gc% | any_th | 3′_th | hairpin | seq |
|---|---|---|---|---|---|---|---|---|
| LEFT-PRIMER | 353 | 20 | 58.88 | 55.00 | 0.00 | 0.00 | 0.00 | GGCCCTATCAGTACAGCAGT |
| | | | | | | | | (SEQ ID NO: 62) |
| RIGHT-PRIMER | 470 | 20 | 59.39 | 50.00 | 0.00 | 0.00 | 0.00 | AGTGCTGGCGAGAGAATGAA |
| | | | | | | | | (SEQ ID NO: 63) |

Vermont-Chromosome 2
ACACAAACTGGCGTAGAAGGGGAAACGGAAATAGGGTCTGACGAGGAAGATAGCA
TAGAGGACGAGGGAAGCAGCACTCTCCCATTAGTCGGCAGCACGTTCGCCAGTAAT
TACCGGAGACAGAAAAATCTCGGAACAGTTTATCCGCAATTCTGAGGAAATCGTCG
TCCGCAAGCTCCGTGCACAGCTAGTAGTAGTCTCCGGTGCGGGGGGGGGCGGAGTG
GTCTCCCACGATACGACGTTGTCTAGATACGTACCCACCTCGCTGTGTGCTCTCTGG
CTATCTGAACGTCCACTCCAGAaggGGCCCTATCAGTACAGCAGTCATAGCCGCACAC
AAGTCCAACGTCCCCCAAACCTCCTGACCACGCAGTCGCCACCGGCGCAGACACTA
TTTCTCGTaggTTCATTCTCTCGCCAGCACTTGTCGGAACAAAGCGGTCTTAGTGGAG
GAAATAGTACGACAGAAAGACTAGTACCACACCAGCTGAGGGAACAAGCAGCCAG
ACATATAGGAAAA (SEQ ID NO: 64)
Vermont-Chromosome 6
TGGAGTTGCAAAAAACAAGGGAAAGGAAAATCAATCAAATTAGAATTAAGGTTTTT
TTTGGACAGTGCAGCGTCAACTCTCCCATTAGTCGGCAGCACGTTCGCCAGTAATTA
CCGGAGACAGAAAAATCTCGGAACAGTTTATCCGCAATTCTGAGGAAATCGTCGTC
CGCAAGCTCCGTGCACAGCTAGTAGTAGTCTCCGGTGCGGGGGGGGGCGGAGTGGT
CTCCCACGATACGACGTTGTCTAGATACGTACCCACCTCGCTGTGTGCTCTCTGGCT
ATCTGAACGTCCACTCCAGAaggGGCCCTATCAGTACAGCAGTCATAGCCGCACACA
AGTCCAACGTCCCCCAAACCTCCTGACCACGCAGTCGCCACCGGCGCAGACACTATT
TCTCGTaggTTCATTCTCTCGCCAGCACTTGTCGGAACAAAGCGGTCTTATGCGCACG
TAATGGCTTCGAAGAAAAAAAGAAGGCAAATACAATGAAGCTGAGATCTTGTTTTA
TCATGAGGGG (SEQ ID NO: 65)
Vermont-Chromosome 14
CAAATAAATTAGGCTCATAACCGTAATTTTATTCGAGACATTTTTGGTTACTTCAAA
ATATTGTTATTATATAAAACTCTCCCATTAGTCGGCAGCACGTTCGCCAGTAATTACC
GGAGACAGAAAAATCTCGGAACAGTTTATCCGCAATTCTGAGGAAATCGTCGTCCG
CAAGCTCCGTGCACAGCTAGTAGTAGTCTCCGGTGCGGGGGGGGGCGGAGTGGTCT
CCCACGATACGACGTTGTCTAGATACGTACCCACCTCGCTGTGTGCTCTCTGGCTAT
CTGAACGTCCACTCCAGAaggGGCCCTATCAGTACAGCAGTCATAGCCGCACACAAG
TCCAACGTCCCCCAAACCTCCTGACCACGCAGTCGCCACCGGCGCAGACACTATTTC
TCGTaggTTCATTCTCTCGCCAGCACTTGTCGGAACAAAGCGGTCTTGATCATATAAA
GTTCTTGGACAAGATTGGATACATTTAGTTTTATTTTTGAAAATCACAAAGATGAAA
CAAAATA (SEQ ID NO: 66)
French Saison
ID1 (PCR Primers)

| OLIGO | start | len | tm | gc% | any_th | 3′_th | hairpin | seq |
|---|---|---|---|---|---|---|---|---|
| LEFT-PRIMER | 79 | 20 | 58.55 | 50.00 | 3.00 | 3.00 | 3.00 | GCGTACAATGCCCTGAAGAA |
| | | | | | | | | (SEQ ID NO: 67) |
| RIGHT-PRIMER | 139 | 20 | 58.82 | 55.00 | 3.00 | 3.00 | 3.00 | CTCCCTGGAAATGCACTTGG |
| | | | | | | | | (SEQ ID NO: 68) |

ID2 (qPCR Primers)

| OLIGO | start | len | tm | gc% | any_th | 3′_th | Hairpin | seq |
|---|---|---|---|---|---|---|---|---|
| LEFT-PRIMER | 308 | 20 | 59.10 | 50.00 | 3.00 | 3.00 | 0.00 | GCGGGTCATCGAAAGGTTA |
| | | | | | | | | SEQ ID NO: 69) |
| RIGHT-PRIMER | 426 | 20 | 58.41 | 50.00 | 3.00 | 3.00 | 0.00 | CACTTGGTCGTTTGGCTGTA |
| | | | | | | | | SEQ ID NO: 70) |

French-Chromosome 2
ACACAAACTGGCGTAGAAGGGGAAACGGAAATAGGGTCTGACGAGGAAGATAGCA
TAGAGGACGAGGGAAGCAGCGCGTACAATGCCCTGAAGAATTACTTCCGTACTGGA
AGCGGATAGCACCAGACTGTAAGCTAACGAACGCCTGTTTGAGGCTCAGTCTGCTA
AATTGGAACCGCGTCGCTCCTAGGCATATTTTGGTGAAAGCACTCTGCCCAAAAGCC
TGTAGAATTCCGGACCGACGCTCTCTTCACTCGAAGATTCCGGGTAAGAAGTTTCAG
CCAGGGCTGTCTCCATTAGAAaggAGCGGGTCATCGAAAGGTTACGTTGGTTGTATCT
GATTAGACGGTAGACATCCAGCTCATCTCTGATTACTAAAGTTCTCCGCCGCTCCAT
CGGGCGaggTACAGCCAAACGACCAAGTGCCAAGTGCATTTCCAGGGAGAGTGGAGG
AAATAGTACGACAGAAAGACTAGTACCACACCAGCTGAGGGAACAAGCAGCCAGA
CATATAGGAAAA (SEQ ID NO: 71)
French-Chromosome 6
TGGAGTTGCAAAAAACAAGGGAAAGGAAAATCAATCAAATTAGAATTAAGGTTTTT
TTTGGACAGTGCAGCGTCAGCGTACAATGCCCTGAAGAATTACTTCCGTACTGGAAG
CGGATAGCACCAGACTGTAAGCTAACGAACGCCTGTTTGAGGCTCAGTCTGCTAAAT
TGGAACCGCGTCGCTCCTAGGCATATTTTGGTGAAAGCACTCTGCCCAAAAGCCTGT
AGAATTCCGGACCGACGCTCTCTTCACTCGAAGATTCCGGGTAAGAAGTTTCAGCCA
GGGCTGTCTCCATTAGAAaggAGCGGGTCATCGAAAGGTTACGTTGGTTGTATCTGAT
TAGACGGTAGACATCCAGCTCATCTCTGATTACTAAAGTTCTCCGCCGCTCCATCGG
GCGaggTACAGCCAAACGACCAAGTGCCAAGTGCATTTCCAGGGAGATGCGCACGTA
ATGGCTTCGAAGAAAAAAAGAAGGCAAATACAATGAAGCTGAGATCTTGTTTTATC
ATGAGGGG (SEQ ID NO: 72)
French-Chromosome 14
CAAATAAATTAGGCTCATAACCGTAATTTTATTCGAGACATTTTTGGTTACTTCAAA
ATATTGTTATTATATAAAGCGTACAATGCCCTGAAGAATTACTTCCGTACTGGAAGC
GGATAGCACCAGACTGTAAGCTAACGAACGCCTGTTTGAGGCTCAGTCTGCTAAATT
GGAACCGCGTCGCTCCTAGGCATATTTTGGTGAAAGCACTCTGCCCAAAAGCCTGTA
GAATTCCGGACCGACGCTCTCTTCACTCGAAGATTCCGGGTAAGAAGTTTCAGCCAG
GGCTGTCTCCATTAGAAaggAGCGGGTCATCGAAAGGTTACGTTGGTTGTATCTGATT
AGACGGTAGACATCCAGCTCATCTCTGATTACTAAAGTTCTCCGCCGCTCCATCGGG
CGaggTACAGCCAAACGACCAAGTGCCAAGTGCATTTCCAGGGAGGATCATATAAAG
TTCTTGGACAAGATTGGATACATTTAGTTTTATTTTTGAAAATCACAAAGATGAAAC
AAAATA (SEQ ID NO: 73)

One or more illustrative embodiments have been described by way of example. It will be understood to persons skilled in the art that a number of variations and modifications can be made without departing from the scope of the invention as defined in the claims.

### REFERENCES

FDA. (2019). Investigation Summary: Factors Potentially Contributing to the Contamination of Romaine Lettuce Implicated in the Fall 2018 Multi-State Outbreak of E. coli O157:H7. Retrieved from https://www.fda.gov/media/120722/download

Food and Drug Regulations C.R.C. c. 870 (2019).

GS1 US. (2013). Integrated Traceability in Fresh Foods: Ripe Opportunity for Real Results. Retrieved from https://www.gslus.org/DesktopModules/Bring2mind/DMX/Download.aspx?Command=Core_Download&EntryId=598.

Introduction of Organisms and Products Altered or Produced Through Genetic Engineering Which Are Plant Pests or Which There Is Reason to Believe Are Plant Pests C.F.R. § 340.1 (2019).

WHO, Foodborne Disease Burden Epidemiology Reference Group. 2015. WHO Estimates of the Global Burden of Foodborne Diseases. Retrieved from https://academicanswers.waldenu.edu/faq/73164.

System of centromeric, episomal, and integrative vectors based on drug resistance markers for Saccharomyces cerevisiae. Christof Taxis and Michael Knop EMBL, Heidelberg, Germany. BioTechniques 40:73-78 (January 2006) doi 10.2144/000112040

Krogerus, K., Magalhães, F., Kuivanen, J. et al. A deletion in the STA1 promoter determines maltotriose and starch utilization in STA1+ Saccharomyces cerevisiae strains. Appl Microbiol Biotechnol 103, 7597-7615 (2019). https://doi.org/10.1007/s00253-019-10021-y

Zheng L, Baumann U, Reymond J L. An efficient one-step site-directed and site-saturation mutagenesis protocol. Nucleic Acids Res. 2004; 32(14):e115. Published 2004 Aug. 10. doi:10.1093/nar/gnh110

Mertens S, Steensels J, G. B, V Kevin J. Rapid Screening Method for Phenolic Off-Flavor (POF) Production in Yeast. J Am Soc Brew Chem. 2017; 75(4):318-23

Mertens S, Gallone B, Steensels J, et al. Reducing phenolic off-flavors through CRISPR-based gene editing of the FDC1 gene in Saccharomyces cerevisiae x Saccharomyces eubayanus hybrid lager beer yeasts [published correction appears in PLoS One. 2019 Oct. 24; 14(10):e0224525]. PLoS One. 2019; 14(1):e0209124. Published 2019 Jan. 9. doi:10.1371/journal.pone.0209124

Mertens S, Gallone B, Steensels J, et al. Correction: Reducing phenolic off-flavors through CRISPR-based gene editing of the FDC1 gene in Saccharomyces cerevisiae x Saccharomyces eubayanus hybrid lager beer yeasts. PLoS One. 2019; 14(10):e0224525. Published 2019 Oct. 24. doi:10.1371/journal.pone.0224525

Bolger A M, Lohse M, Usadel B. Trimmomatic: a flexible trimmer for Illumina sequence data. Bioinformatics. 2014 Aug. 1; 30(15):2114-20. doi: 10.1093/bioinformatics/btu170. Epub 2014 Apr. 1. PMID: 24695404; PMCID: PMC4103590.

Chiang C, Layer R M, Faust G G, Lindberg M R, Rose D B, Garrison E P, Marth G T, Quinlan A R, Hall I M. SpeedSeq: ultra-fast personal genome analysis and interpretation. Nat Methods. 2015 October; 12(10):966-8. doi: 10.1038/nmeth.3505. Epub 2015 Aug. 10. PMID: 26258291; PMCID: PMC4589466.

Garcia-Alcalde F, Okonechnikov K, Carbonell J, Cruz L M, Götz S, Tarazona S, Dopazo J, Meyer T F, Conesa A. Qualimap: evaluating next-generation sequencing alignment data. Bioinformatics. 2012 Oct. 15; 28(20):2678-9. doi: 10.1093/bioinformatics/bts503. Epub 2012 Aug. 22. PMID: 22914218.

Erik Garrison and Gabor Marth 2012. Haplotype-based variant detection from short-read sequencing

Li H, Handsaker B, Wysoker A, Fennell T, Ruan J, Homer N, Marth G, Abecasis G, Durbin R; 1000 Genome Project Data Processing Subgroup. The Sequence Alignment/Map format and SAMtools. Bioinformatics. 2009 Aug. 15; 25(16):2078-9. doi: 10.1093/bioinformatics/btp352. Epub 2009 Jun. 8. PMID: 19505943; PMCID: PMC2723002

Cingolani P, Platts A, Wang le L, Coon M, Nguyen T, Wang L, Land S J, Lu X, Ruden D M. A program for annotating and predicting the effects of single nucleotide polymorphisms, SnpEff: SNPs in the genome of Drosophila melanogaster strain w1118; iso-2; iso-3. Fly (Austin). 2012 April-June; 6(2):80-92. doi: 10.4161/fly.19695. PMID: 22728672; PMCID: PMC3679285.

Boeva V, Popova T, Bleakley K, et al. Control-FREEC: a tool for assessing copy number and allelic content using next-generation sequencing data. Bioinformatics. 2012; 28(3):423-425. doi:10.1093/bioinformatics/btr670

Quinlan A R, Hall I M. BEDTools: a flexible suite of utilities for comparing genomic features. Bioinformatics. 2010 Mar. 15; 26(6):841-2. doi: 10.1093/bioinformatics/btq033. Epub 2010 Jan. 28. PMID: 20110278; PMCID: PMC2832824

All references cited herein and elsewhere in the specification are herein incorporated by reference in their entireties.

What is claimed is:

1. A method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the biological material.

2. The method of claim 1, wherein the biological material comprises a plant-based material, a fungus-based material, an animal-based material, a virus-based material, or a bacterial-based material.

3. A method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and comparing the sequence of the DNA unique identifier sequence with a database to confirm that the DNA unique identifier sequence is not already used in the database;
providing an indication of acceptability to produce a biological material from the biological entity, the biological material comprising genomic DNA from the biological entity; and
inputting the sequence of the at least one DNA unique identifier sequence into a database entry of the database, and associating the DNA unique identifier sequence with identification and/or tracking information for the biological material;
thereby providing traceability of the biological material by reading the DNA unique identifier sequence in the biological material and retrieving the corresponding database entry providing the identification and/or tracking information for the biological material.

4. The method of claim 3, further comprising inserting at least one DNA unique identifier sequence within the genomic DNA of a biological entity, or modifying a pre-existing identifier sequence within the genomic DNA of a biological entity by gene editing to create a DNA unique identifier sequence within the genomic DNA of the biological entity, thereby providing identification thereof.

5. The method of claim 4, further comprising providing the at least one DNA unique identifier sequence for the insertion within the genomic DNA of the biological entity.

6. The method according to any one of claims 3-5, wherein the biological material comprises a plant-based material, a fungus-based material, an animal-based material, a virus-based material, or a bacterial-based material.

7. The method of claim any one of claims 3-6, wherein the biological entity comprises a plant cell, a fungal cell, an animal cell, a virus, or a bacterial cell.

8. The method of claim any one of claims 3-7, wherein producing a biological material from the biological entity comprising propagating the biological entity.

9. The method of any one of claims 3-8, wherein the DNA unique identifier sequence is from a randomized pool of DNA unique identifier sequences.

10. The method of any one of claims 3-9, wherein reading the DNA unique identifier sequence in the biological material and retrieving the corresponding database entry comprises:
receiving or providing a sample comprising genomic DNA from the biological material;
amplifying the at least one DNA unique identifier sequence within the genomic DNA from the biological material and sequencing the DNA unique identifier sequence; and
comparing the DNA unique identifier sequence to the database and retrieving the database entry corresponding with the DNA unique identifier sequence, the database entry providing identification and/or tracking information for the biological material.

11. The method of any one of claims 1-10, wherein the DNA unique identifier sequence comprises a unique nucleotide sequence inserted into an intergenic region of the genomic DNA.

12. The method of any one of claims 1-11, wherein the DNA unique identifier sequence comprises a sequence of up to about 1500 nt in length; up to about 1000 nt in length; about 200 nt to about 600 nt in length; about 200 nt to about 400 nt in length; or about 400 nt to about 600 nt in length.

13. The method of any one of claims 1-12, wherein the DNA unique identifier sequence is flanked by one or more primer annealing sequences for PCR amplification of the DNA unique identifier sequence, sequencing of the DNA unique identifier sequence, or both.

14. The method of any one of claims 1-13, wherein the biological material comprises a food.

15. The method of any one of claims 1-14, wherein the identification and/or tracking information of the database entry comprises supply chain information for the biological material.

16. The method of any one of claims 1-15, wherein the identification and/or tracking information of the database entry comprises source-of-origin information for the biological material.

17. The method of any one of claims 1-16, wherein the identification and/or tracking information of the database entry comprises grower, region, batch, lot, date, or other relevant supply chain information, or any combinations thereof.

18. The method of any one of claims 1-17, wherein a cassette is incorporated into the genomic DNA, wherein the cassette comprises the DNA unique identifier sequence flanked by one or more primer annealing sequences for PCR amplification of the DNA unique identifier sequence, sequencing of the DNA unique identifier sequence, or both.

19. The method of any one of claims 1-18, wherein the DNA unique identifier sequence is a random sequence derived from a randomized pool of nucleic acid sequences of up to about 1500 nt in length; up to about 1000 nt in length; about 200 nt to about 600 nt in length; about 200 nt to about 400 nt in length; or about 400 nt to about 600 nt in length.

20. An oligonucleotide comprising a DNA unique identifier sequence flanked by one or more primer annealing sequences for PCR amplification of the DNA unique identifier sequence, sequencing of the DNA unique identifier sequence, or both.

21. The oligonucleotide of claim 20, wherein the DNA unique identifier sequence comprises a random sequence of up to about 1500 nt in length; up to about 1000 nt in length; about 200 nt to about 600 nt in length; about 200 nt to about 400 nt in length; or about 400 nt to about 600 nt in length.

22. A cassette comprising the oligonucleotide of claim 20 or 21.

23. A cell or virus comprising the oligonucleotide of claim 20 or 21, or the cassette of claim 22, incorporated into the genome of the cell or virus.

24. A cell or virus comprising a DNA unique identifier sequence incorporated into the genome of the cell or virus.

25. The cell or virus of claim 23 or 24, wherein the DNA unique identifier sequence is incorporated into an intergenic region of the genomic DNA of the cell or virus.

26. The cell or virus of any one of claims 23-25, wherein the cell is a plant cell, a fungal cell, an animal cell, or a bacterial cell.

27. A kit comprising any one or more of:
a DNA unique identifier sequence;
a randomized pool of DNA unique identifier sequences;
an oligonucleotide as defined in claim 20 or 21;
a cassette as defined in claim 22;
one or more primer pairs for amplifying and/or sequencing a DNA unique identifier sequence;
a buffer;
a polymerase; or
instructions for performing a method according to any one of claims 1-19.

28. A method of identifying a biological material, the method comprising:
receiving at a computing device a DNA-unique identifier sequence (DUID) extracted from a known biological material;
searching at the computing device a DUID database storing a plurality of DUIDs in association with respective biological material information for a match to the received DUID;
if the search of the DUID database fails to provide a match to the received DUID, storing in the DUID database the received DUID in association with biological material information associated with the known biological material;
subsequent to storing the received DUID and with information associated with the known biological material in the DUID database, receiving at the computing device a query DUID extracted from an unknown biological material;
searching at the computing device the DUID database for a match to the received query DUID; and
if the search of the DUID provides a match to the received query DUID, returning in response to the received query DUID the biological information stored in association with the DUID matching the query DUID.

29. The method of claim 28, wherein searching the DUID database for a match to the received DUID comprises:
searching the DUID database for an exact match to the received DUID; and
if an exact match is not found, performing an alignment/identity search for DUIDs stored in the DUID database that are a close match to the received DUID.

30. The method of claim 28 or 29, wherein searching the DUID database for a match to the query DUID comprises:
searching the DUID database for an exact match to the query DUID; and
if an exact match is not found, performing an alignment/identity search for DUIDs stored in the DUID database that are a close match to the query DUID.

31. The method of claim 30, further comprising:
if the search provides a close match to the query DUID, storing the query DUID in association with the DUID that is a close match to the query DUID.

32. A computing system for identifying a biological material, the system comprising:
a processing unit capable of executing instructions; and
a memory unit storing instructions, which when executed by the processing unit configure the computing system to perform the method according to any one of claims 28-31.

33. A computer readable memory, having instructions stored thereon, which when executed by a processing unit of a computing system configure the system to perform the method according to any one of claims 28-31.

34. A method for identifying a biological material, said method comprising:
receiving or providing a sample comprising genomic DNA from the biological material;
amplifying at least one DNA unique identifier sequence within the genomic DNA from the biological material and sequencing the DNA unique identifier sequence; and
decoding or decrypting identification and/or tracking information for the biological material stored in the DNA unique identifier sequence.

35. A method for providing traceability of biological material, said method comprising:
determining the sequence of at least one DNA unique identifier sequence within the genomic DNA of a biological entity;
validating identification of the biological entity by: verifying presence of the DNA unique identifier sequence in the genomic DNA; and decoding or decrypting identification and/or tracking information stored in the DNA unique identifier sequence to verify the DNA unique identifier sequence; and
providing an indication of acceptability to produce a biological material from the biological entity, the biological material comprising genomic DNA from the biological entity;
thereby providing traceability of the biological material by reading the DNA unique identifier sequence in the biological material and decoding or decrypting information stored in the DNA unique identifier sequence, providing identification and/or tracking information for the biological material.

36. A method of identifying a biological material, the method comprising:
receiving at a computing device a DNA-unique identifier sequence (DUID) extracted from an unknown biological material; and
decoding or decrypting identification and/or tracking information for the unknown biological material stored in the DNA unique identifier sequence.

37. A cassette comprising a DNA unique identifier sequence, the DNA unique identifier sequence flanked by at least one 5′ primer annealing sequence and at least one 3′ primer annealing sequence for amplification of the DNA unique identifier sequence, sequencing of the DNA unique identifier sequence, or both.

38. The cassette of claim 37, wherein the DNA unique identifier sequence is flanked by two 5′ primer annealing sequences and two 3′ primer annealing sequences to allow for amplification of the DNA unique identifier sequence by nested PCR.

39. The cassette of claim 38, wherein the two 5′ primer annealing sequences are partially overlapping; wherein the two 3′ primer annealing sequences are partially overlapping; or both.

40. The cassette of any one of claims 37-39, wherein the cassette further comprises a sequencing primer annealing sequence located 5′ to the DNA unique identifier sequence for sequencing of the DNA unique identifier sequence.

41. The cassette of claim 40, wherein the sequencing primer annealing sequence is positioned between two 5′ primer annealing sequences.

42. The cassette of claim 41, wherein the sequencing primer annealing sequence at least partially overlaps with one or both of the two 5′ primer annealing sequences.

43. The cassette of claim 41, wherein the two 5′ primer annealing sequences are partially overlapping, and wherein at least a portion of the sequencing primer annealing sequence is positioned at the overlap.

44. The cassette of any one of claims 37-43, wherein the cassette sequence is up to about 1500 nt in length; up to about 1000 nt in length; about 200 nt to about 600 nt in length; about 200 nt to about 400 nt in length; or about 400 nt to about 600 nt in length.

45. The cassette of any one of claims 37-44, wherein the primer annealing sequences are not naturally occurring in the genome of a target biological entity.

46. A composition comprising a plurality of cassettes as defined in any one of claims 37-45, each cassette comprising the same primer annealing sequences, and each cassette comprising a randomized DNA unique identifier sequence.

47. A composition comprising a plurality of cassettes as defined in any one of claims 40-43, each cassette comprising the same primer annealing sequences and the same sequencing primer annealing sequence, and each cassette comprising a randomized DNA unique identifier sequence.

48. A method for providing traceability of biological material, said method comprising:
inserting at least one DNA unique identifier sequence within the genomic DNA of a biological entity for use in preparing the biological material.

49. The method of claim 48, wherein the DNA unique identifier sequence is inserted as a cassette according to any one of claims 37-45.

50. The method of claim 48 or 49, further comprising a step of determining the sequence of the least one DNA unique identifier sequence within the genomic DNA of the biological entity.

51. The method of any one of claims 48-50, further comprising a step of validating identification of the biological entity by: verifying presence of the DNA unique identifier sequence in the genomic DNA; and comparing the sequence of the DNA unique identifier sequence with a database to confirm that the DNA unique identifier sequence is not already used in the database.

52. The method of any one of claims 48-51, further comprising a step of:
producing the biological material from the biological entity, the biological material comprising genomic DNA from the biological entity; and/or
providing an indication of acceptability to produce the biological material from the biological entity, the biological material comprising genomic DNA from the biological entity.

53. The method of any one of claims 48-52, further comprising a step of inputting the sequence of the at least one DNA unique identifier sequence into a database entry, and associating the DNA unique identifier sequence with identification and/or tracking information for the biological entity and/or biological material.

54. The method of claim 53, further comprising a step of:
providing traceability of the biological entity and/or biological material by reading the DNA unique identifier sequence in the biological entity and/or biological material and retrieving the corresponding database entry providing the identification and/or tracking information for the biological entity and/or biological material.

55. A plasmid or expression vector comprising an oligonucleotide according to any one of claims 20-21, or a cassette according to any one of claim 22, 37-44, or 45.

56. A method for providing traceability of a product of interest, said method comprising:
receiving or providing a sample from the product of interest, the sample comprising genomic DNA from a biological material part of, mixed with, or otherwise associated with the product of interest;
amplifying at least one DNA unique identifier sequence within the genomic DNA from the biological material and sequencing the DNA unique identifier sequence; and
searching for the DNA unique identifier sequence in a database and retrieving a database entry corresponding with the DNA unique identifier sequence, the database entry providing identification and/or tracking information for the product of interest.

57. The method of claim 56, wherein the method comprises introducing or adding the biological material to the product of interest, the biological material comprising at least one DNA unique identifier sequence as part of its genomic material.

58. The method of claim 56 or 57, wherein the identification and/or tracking information of the database entry comprises supply chain information for the product of interest.

59. The method of any one of claims 56-58, wherein the product of interest comprises food, an agricultural product, a pharmaceutical drug, a retail product, textiles, commodities, chemicals, or another supply chain item.


SEQUENCE LISTING (73 sequences)

SEQ ID NO: 1 - DNA, 296 bp, Artificial Sequence
  Random DNA UID Sequence
cgcctaagctcctgtgacgggagagagaacggattcggtggtagaactcgcccgtgcagacataattgcggattctcgggggcgtagatgcctaagaatacctcaacgcttctgcatgatggtactcactctagttgtttaatttaccacgccagtagactatgggcaagtcagcgcgcaagagacactcaccgttgaattaacagcaccacccctttactccacgcgaaaggcggattatcgtatcttttttgtggcgaatttatgtatctctccttgaggaagcagagcagtcc

SEQ ID NO: 2 - DNA, 50 bp, Artificial Sequence
  DUID#1
acaacggtcgtatgtatgcactaggtcaacaataggacatagccttgtag

SEQ ID NO: 3 - DNA, 50 bp, Artificial Sequence
  DUID#2
acaacggtcgttgtgttccgacaggctagcatattatcctaaggcgttac

SEQ ID NO: 4 - DNA, 50 bp, Artificial Sequence
  DUID#3
acaacggtcgtaccgtcggatttgctatagcccctgaacgctacatgtac

SEQ ID NO: 5 - DNA, 19 bp, Artificial Sequence
  Primer, Chr6_DUID
cattccgcctgacctggag

SEQ ID NO: 6 - DNA, 21 bp, Artificial Sequence
  Primer, Euch_DUID F
cattccgcctgaccccttaat

SEQ ID NO: 7 - DNA, 19 bp, Artificial Sequence
  Primer, DUID_synth R
cactgagcctccacctagc

SEQ ID NO: 8 - DNA, 20 bp, Artificial Sequence
  Primer, Chr6_Seq F
aagcgtaattccgaaaggca

SEQ ID NO: 9 - DNA, 20 bp, Artificial Sequence
  Primer, Chr6_Seq R
tgcatacgcttctctcgact

SEQ ID NO: 10 - DNA, 25 bp, Artificial Sequence
  Primer, Euch_Seq F
cagaaatggacaaggagatatgtga

SEQ ID NO: 11 - DNA, 21 bp, Artificial Sequence
  Primer, Euch_Seq R
ttgagtacctggccaatggag

SEQ ID NO: 12 - DNA, 19 bp, Artificial Sequence
  Primer, S288C_recall F
gctgatggtttaggcgtac

SEQ ID NO: 13 - DNA, 20 bp, Artificial Sequence
  Primer, S288C_recall R
ccctggaaatgcacttggtc

SEQ ID NO: 14 - DNA, 20 bp, Artificial Sequence
  Primer, S288C_qPCR F
tggtcgtttggctgtagaga

SEQ ID NO: 15 - DNA, 20 bp, Artificial Sequence
  Primer, S288C_qPCR R
cgtatagagcgggtcatcga

SEQ ID NO: 16 - DNA, 20 bp, Artificial Sequence
  Primer, Verm_recall F
actctcccattagtcggcag

SEQ ID NO: 17 - DNA, 20 bp, Artificial Sequence
  Primer, Verm_recall R
aagaccgctttgttccgaca

SEQ ID NO: 18 - DNA, 20 bp, Artificial Sequence
  Primer, Verm_qPCR F
ggccctatcagtacagcagt

SEQ ID NO: 19 - DNA, 20 bp, Artificial Sequence
  Primer, Verm_qPCR R
agtgctggcgagagaatgaa

SEQ ID NO: 20 - DNA, 20 bp, Artificial Sequence
  Primer, FrenSais_recall F
gcgtacaatgccctgaagaa

SEQ ID NO: 21 - DNA, 20 bp, Artificial Sequence
  Primer, FrenSais_recall R
ctccctggaaatgcacttgg

SEQ ID NO: 22 - DNA, 20 bp, Artificial Sequence
  Primer, FrenSais_qPCR F
agcgggtcatcgaaaggtta

SEQ ID NO: 23 - DNA, 20 bp, Artificial Sequence
  Primer, FrenSais_qPCR R
cacttggtcgtttggctgta

SEQ ID NO: 24 - DNA, 54 bp, Artificial Sequence
  Primer, Chr6_1 F
agttgcaaaaaacaagggaagttttagagctagaaatagcaagttaaaataagg

SEQ ID NO: 25 - DNA, 43 bp, Artificial Sequence
  Primer, Chr6_1 R
tcccttgttttttgcaactgatcatttatctttcactgcggag

SEQ ID NO: 26 - DNA, 54 bp, Artificial Sequence
  Primer, Chr6_2 F
gagatcttgttttatcatgagttttagagctagaaatagcaagttaaaataagg

SEQ ID NO: 27 - DNA, 43 bp, Artificial Sequence
  Primer, Chr6_2 R
catgataaaacaagatctcgatcatttatctttcactgcggag

SEQ ID NO: 28 - DNA, 54 bp, Artificial Sequence
  Primer, Chr6_3 F
agatcttgttttatcatgaggttttagagctagaaatagcaagttaaaataagg

SEQ ID NO: 29 - DNA, 43 bp, Artificial Sequence
  Primer, Chr6_3 R
tcatgataaaacaagatctgatcatttatctttcactgcggag

SEQ ID NO: 30 - DNA, 54 bp, Artificial Sequence
  Primer, Euch_1 F
atactaagtcaacatcaagggttttagagctagaaatagcaagttaaaataagg

SEQ ID NO: 31 - DNA, 44 bp, Artificial Sequence
  Primer, Euch_1 R
ccttgatgttgacttagtatgatcatttatctttcactgcggag

SEQ ID NO: 32 - DNA, 54 bp, Artificial Sequence
  Primer, Euch_2 F
gaaatactaagtcaacatcagttttagagctagaaatagcaagttaaaataagg

SEQ ID NO: 33 - DNA, 44 bp, Artificial Sequence
  Primer, Euch_2 R
tgatgttgacttagtatttcgatcatttatctttcactgcggag

SEQ ID NO: 34 - DNA, 54 bp, Artificial Sequence
  Primer, Euch_3 F
tcttggcttttacaaccgaggttttagagctagaaatagcaagttaaaataagg

SEQ ID NO: 35 - DNA, 44 bp, Artificial Sequence
  Primer, Euch_3 R
ctcggttgtaaaagccaagagatcatttatctttcactgcggag

SEQ ID NO: 36 - DNA, 28 bp, Artificial Sequence
  Primer, Chr6_1 mut
caagggaaacgaaaatcaatcaaattag

SEQ ID NO: 37 - DNA, 24 bp, Artificial Sequence
  Primer, Chr6_1 mut R
gattgattttcgtttcccttgttt

SEQ ID NO: 38 - DNA, 28 bp, Artificial Sequence
  Primer, Chr6_2_3 mut R
cctccacctagcctccgctcatgataaa

SEQ ID NO: 39 - DNA, 20 bp, Artificial Sequence
  Primer, Chr6_Vector R
gctgcagcccctcatgataa

SEQ ID NO: 40 - DNA, 31 bp, Artificial Sequence
  Primer, Euch_1 mut F
gaggaaatactaagtcaacatcaaggtcgca

SEQ ID NO: 41 - DNA, 35 bp, Artificial Sequence
  Primer, Euch_1 mut R
tgcgaccttgatgttgacttagtatttcctctcgg

SEQ ID NO: 42 - DNA, 22 bp, Artificial Sequence
  Primer, Euch_2 mut F
ctaagtcaacatcaacgtggca

SEQ ID NO: 43 - DNA, 29 bp, Artificial Sequence
  Primer, Euch_2 mut R
tgccacgttgatgttgacttagtatttcc

SEQ ID NO: 44 - DNA, 31 bp, Artificial Sequence
  Primer, Euch_3 mut F
tacaaccgagacgaaatactaagtcaacatc

SEQ ID NO: 45 - DNA, 30 bp, Artificial Sequence
  Primer, Euch_3 mut R
cttagtatttcgtctcggttgtaaaagcca

SEQ ID NO: 46 - DNA, 18 bp, Artificial Sequence
  Primer, Euch_Vector R
gggctgcagtcagcagat

SEQ ID NO: 47 - DNA, 75 bp, Artificial Sequence
  Yeast chromosomal integration Sites, ChrII809650..809799, Left Homology
acacaaactggcgtagaaggggaaacggaaatagggtctgacgaggaagatagcatagaggacgagggaagcagc

SEQ ID NO: 48 - DNA, 75 bp, Artificial Sequence
  Yeast chromosomal integration Sites, ChrII809650..809799, Right Homology
agtggaggaaatagtacgacagaaagactagtaccacaccagctgagggaacaagcagccagacatataggaaaa

SEQ ID NO: 49 - DNA, 75 bp, Artificial Sequence
  Yeast chromosomal integration Sites, ChrVI261123..261272, Left Homology
tggagttgcaaaaaacaagggaaaggaaaatcaatcaaattagaattaaggttttttttggacagtgcagcgtca

SEQ ID NO: 50 - DNA, 75 bp, Artificial Sequence
  Yeast chromosomal integration Sites, ChrVI261123..261272, Right Homology
atgcgcacgtaatggcttcgaagaaaaaaagaaggcaaatacaatgaagctgagatcttgttttatcatgagggg

SEQ ID NO: 51 - DNA, 75 bp, Artificial Sequence
  Yeast chromosomal integration Sites, ChrXIV764201..764350, Left Homology
caaataaattaggctcataaccgtaattttattcgagacatttttggttacttcaaaatattgttattatataaa

SEQ ID NO: 52 - DNA, 75 bp, Artificial Sequence
  Yeast chromosomal integration Sites, ChrXIV764201..764350, Right Homology
gatcatataaagttcttggacaagattggatacatttagttttatttttgaaaatcacaaagatgaaacaaaata

SEQ ID NO: 53 - DNA, 20 bp, Artificial Sequence
  S288c, ID1 (PCR Primers), Left Primer
gctgatggtttaggcgtaca

SEQ ID NO: 54 - DNA, 20 bp, Artificial Sequence
  S288c, ID1 (PCR Primers), Right Primer
ccctggaaatgcacttggtc

SEQ ID NO: 55 - DNA, 19 bp, Artificial Sequence
  S288c, ID2 (qPCR Primers), left primer
cgtatagagcgggtcatcg

SEQ ID NO: 56 - DNA, 20 bp, Artificial Sequence
  S288c, ID2 (qPCR Primers), right primer
tggtcgtttggctgtagaga

SEQ ID NO: 57 - DNA, 520 bp, Artificial Sequence
  s288c - Chromosome 2
acacaaactggcgtagaaggggaaacggaaatagggtctgacgaggaagatagcatagaggacgagggaagcagcgctgatggtttaggcgtacacgagatcctggttcaacgcgctgcaaacctaccctgctccaaactgctgttcaacgccactctaactggcaggcaaattattagtttctaagttccccaggtgctgaagagcagtcattcaacgccctcagatcatcccggcaagttggctggcgcgtttgtccggaggatcgtgtcgtacaacaaccatctgactatcaaccctccaggcgtatagagcgggtcatcgatgcgctcagggaacaacaacgataggcctgcggctggtcaccatcgggaagttttgctggagatctgctgctgtaggaggtctctacagccaaacgaccagaccaagtgcatttccagggagtggaggaaatagtacgacagaaagactagtaccacaccagctgagggaacaagcagccagacatataggaaaa

SEQ ID NO: 58 - DNA, 520 bp, Artificial Sequence
  s288c - Chromosome 6
tggagttgcaaaaaacaagggaaaggaaaatcaatcaaattagaattaaggttttttttggacagtgcagcgtcagctgatggtttaggcgtacacgagatcctggttcaacgcgctgcaaacctaccctgctccaaactgctgttcaacgccactctaactggcaggcaaattattagtttctaagttccccaggtgctgaagagcagtcattcaacgccctcagatcatcccggcaagttggctggcgcgtttgtccggaggatcgtgtcgtacaacaaccatctgactatcaaccctccaggcgtatagagcgggtcatcgatgcgctcagggaacaacaacgataggcctgcggctggtcaccatcgggaagttttgctggagatctgctgctgtaggaggtctctacagccaaacgaccagaccaagtgcatttccagggatgcgcacgtaatggcttcgaagaaaaaaagaaggcaaatacaatgaagctgagatcttgttttatcatgagggg

SEQ ID NO: 59 - DNA, 520 bp, Artificial Sequence
  s288c - Chromosome 14
caaataaattaggctcataaccgtaattttattcgagacatttttggttacttcaaaatattgttattatataaagctgatggtttaggcgtacacgagatcctggttcaacgcgctgcaaacctaccctgctccaaactgctgttcaacgccactctaactggcaggcaaattattagtttctaagttccccaggtgctgaagagcagtcattcaacgccctcagatcatcccggcaagttggctggcgcgtttgtccggaggatcgtgtcgtacaacaaccatctgactatcaaccctccaggcgtatagagcgggtcatcgatgcgctcagggaacaacaacgataggcctgcggctggtcaccatcgggaagttttgctggagatctgctgctgtaggaggtctctacagccaaacgaccagaccaagtgcatttccaggggatcatataaagttcttggacaagattggatacatttagttttatttttgaaaatcacaaagatgaaacaaaata

SEQ ID NO: 60 - DNA, 20 bp, Artificial Sequence
  Vermont, ID1 (PCR), left primer
actctcccattagtcggcag

SEQ ID NO: 61 - DNA, 20 bp, Artificial Sequence
  Vermont, ID1 (PCR), right primer
aagaccgctttgttccgaca

SEQ ID NO: 62 - DNA, 20 bp, Artificial Sequence
  Vermont, ID2 (qPCR Primers), left primer
ggccctatcagtacagcagt

SEQ ID NO: 63 - DNA, 20 bp, Artificial Sequence
  Vermont, ID2 (qPCR Primers), right primer
agtgctggcgagagaatgaa

SEQ ID NO: 64 - DNA, 520 bp, Artificial Sequence
  Vermont - Chromosome 2
acacaaactggcgtagaaggggaaacggaaatagggtctgacgaggaagatagcatagaggacgagggaagcagcactctcccattagtcggcagcacgttcgccagtaattaccggagacagaaaaatctcggaacagtttatccgcaattctgaggaaatcgtcgtccgcaagctccgtgcacagctagtagtagtctccggtgcgggggggggcggagtggtctcccacgatacgacgttgtctagatacgtacccacctcgctgtgtgctctctggctatctgaacgtccactccagaaggggccctatcagtacagcagtcatagccgcacacaagtccaacgtcccccaaacctcctgaccacgcagtcgccaccggcgcagacactatttctcgtaggttcattctctcgccagcacttgtcggaacaaagcggtcttagtggaggaaatagtacgacagaaagactagtaccacaccagctgagggaacaagcagccagacatataggaaaa

SEQ ID NO: 65 - DNA, 520 bp, Artificial Sequence
  Vermont - Chromosome 6
tggagttgcaaaaaacaagggaaaggaaaatcaatcaaattagaattaaggttttttttggacagtgcagcgtcaactctcccattagtcggcagcacgttcgccagtaattaccggagacagaaaaatctcggaacagtttatccgcaattctgaggaaatcgtcgtccgcaagctccgtgcacagctagtagtagtctccggtgcgggggggggcggagtggtctcccacgatacgacgttgtctagatacgtacccacctcgctgtgtgctctctggctatctgaacgtccactccagaaggggccctatcagtacagcagtcatagccgcacacaagtccaacgtcccccaaacctcctgaccacgcagtcgccaccggcgcagacactatttctcgtaggttcattctctcgccagcacttgtcggaacaaagcggtcttatgcgcacgtaatggcttcgaagaaaaaaagaaggcaaatacaatgaagctgagatcttgttttatcatgagggg

SEQ ID NO: 66 - DNA, 520 bp, Artificial Sequence
  Vermont - Chromosome 14
caaataaattaggctcataaccgtaattttattcgagacatttttggttacttcaaaatattgttattatataaaactctcccattagtcggcagcacgttcgccagtaattaccggagacagaaaaatctcggaacagtttatccgcaattctgaggaaatcgtcgtccgcaagctccgtgcacagctagtagtagtctccggtgcgggggggggcggagtggtctcccacgatacgacgttgtctagatacgtacccacctcgctgtgtgctctctggctatctgaacgtccactccagaaggggccctatcagtacagcagtcatagccgcacacaagtccaacgtcccccaaacctcctgaccacgcagtcgccaccggcgcagacactatttctcgtaggttcattctctcgccagcacttgtcggaacaaagcggtcttgatcatataaagttcttggacaagattggatacatttagttttatttttgaaaatcacaaagatgaaacaaaata

SEQ ID NO: 67 - DNA, 20 bp, Artificial Sequence
  French Saison, ID1 (PCR Primers), left primer
gcgtacaatgccctgaagaa

SEQ ID NO: 68 - DNA, 20 bp, Artificial Sequence
  French Saison, ID1 (PCR Primers), right primer
ctccctggaaatgcacttgg

SEQ ID NO: 69 - DNA, 20 bp, Artificial Sequence
  French Saison, ID2 (qPCR Primers), left primer
agcgggtcatcgaaaggtta

SEQ ID NO: 70 - DNA, 20 bp, Artificial Sequence
  French Saison, ID2 (qPCR Primers), right primer
cacttggtcgtttggctgta

SEQ ID NO: 71 - DNA, 520 bp, Artificial Sequence
  French - Chromosome 2
acacaaactggcgtagaaggggaaacggaaatagggtctgacgaggaagatagcatagaggacgagggaagcagcgcgtacaatgccctgaagaattacttccgtactggaagcggatagcaccagactgtaagctaacgaacgcctgtttgaggctcagtctgctaaattggaaccgcgtcgctcctaggcatattttggtgaaagcactctgcccaaaagcctgtagaattccggaccgacgctctcttcactcgaagattccgggtaagaagtttcagccagggctgtctccattagaaaggagcgggtcatcgaaaggttacgttggttgtatctgattagacggtagacatccagctcatctctgattactaaagttctccgccgctccatcgggcgaggtacagccaaacgaccaagtgccaagtgcatttccagggagagtggaggaaatagtacgacagaaagactagtaccacaccagctgagggaacaagcagccagacatataggaaaa

SEQ ID NO: 72 - DNA, 520 bp, Artificial Sequence
  French - Chromosome 6
tggagttgcaaaaaacaagggaaaggaaaatcaatcaaattagaattaaggttttttttggacagtgcagcgtcagcgtacaatgccctgaagaattacttccgtactggaagcggatagcaccagactgtaagctaacgaacgcctgtttgaggctcagtctgctaaattggaaccgcgtcgctcctaggcatattttggtgaaagcactctgcccaaaagcctgtagaattccggaccgacgctctcttcactcgaagattccgggtaagaagtttcagccagggctgtctccattagaaaggagcgggtcatcgaaaggttacgttggttgtatctgattagacggtagacatccagctcatctctgattactaaagttctccgccgctccatcgggcgaggtacagccaaacgaccaagtgccaagtgcatttccagggagatgcgcacgtaatggcttcgaagaaaaaaagaaggcaaatacaatgaagctgagatcttgttttatcatgagggg

SEQ ID NO: 73 - DNA, 520 bp, Artificial Sequence
  French - Chromosome 14
caaataaattaggctcataaccgtaattttattcgagacatttttggttacttcaaaatattgttattatataaagcgtacaatgccctgaagaattacttccgtactggaagcggatagcaccagactgtaagctaacgaacgcctgtttgaggctcagtctgctaaattggaaccgcgtcgctcctaggcatattttggtgaaagcactctgcccaaaagcctgtagaattccggaccgacgctctcttcactcgaagattccgggtaagaagtttcagccagggctgtctccattagaaaggagcgggtcatcgaaaggttacgttggttgtatctgattagacggtagacatccagctcatctctgattactaaagttctccgccgctccatcgggcgaggtacagccaaacgaccaagtgccaagtgcatttccagggaggatcatataaagttcttggacaagattggatacatttagttttatttttgaaaatcacaaagatgaaacaaaata